(19) United States
(12) Patent Application Publication
(10) Pub. No.: US 2015/0363555 A1
(43) Pub. Date: Dec. 17, 2015

(54) HEALTH INFORMATION SYSTEM

(71) Applicant: Jason T. Studsrud, Minnetonka Beach, MN (US)

(72) Inventor: Jason T. Studsrud, Minnetonka Beach, MN (US)

(21) Appl. No.: 14/740,058

(22) Filed: Jun. 15, 2015

Related U.S. Application Data
(60) Provisional application No. 62/133,876, filed on Mar. 16, 2015.
(60) Provisional application No. 62/012,965, filed on Jun. 16, 2014.

(51) Int. Cl. G06F 19/00; G06Q 10/06

(52) U.S. Cl. CPC G06F 19/327; G06Q 10/0633

(57) ABSTRACT

This document generally describes computer-based technology to provide health information systems that can be customized for any healthcare specialty and/or application. For example, the disclosed computer-based technology can be used in specialties such as imaging centers, radiology group practices, and/or other ambulatory clinics, and can be used for business, financial, and/or clinical applications (e.g., practice management (PM), radiology information systems (RIS), electronic medical records (EMR), electronic health records (EHR)).

### CROSS-REFERENCE TO RELATED APPLICATIONS

This application claims the benefit of U.S. Provisional Application Ser. No. 62/133,876, entitled “HEALTH INFORMATION SYSTEM” and filed Mar. 16, 2015, the entire contents of which are incorporated by reference herein, and also claims the benefit of U.S. Provisional Application Ser. No. 62/012,965, entitled “HEALTH INFORMATION SYSTEM” and filed Jun. 16, 2014, the entire contents of which are also incorporated by reference herein.

### TECHNICAL FIELD

This document generally describes computer-based technology for health information systems.

### BACKGROUND

Computer systems have been developed to manage various aspects of medical practices in the health care field, including computer systems to manage scheduling, billing, and medical records. For example, computer systems have been developed through which doctors and others in the medical field can chart, enter notes, and track information related to patient care.

### SUMMARY

This document generally describes computer-based technology to provide health information systems that can be customized for any healthcare specialty and/or application. For example, the disclosed computer-based technology can be used in various specialized areas of medical practice, such as imaging centers, radiology group practices, and/or other ambulatory clinics, and can be used for business, financial, and/or clinical applications (e.g., practice management (PM), radiology information systems (RIS), electronic medical records (EMR), electronic health records (EHR)), to list just a few examples.

Additionally, the disclosed computer-based technology can serve as a master database that collects and manages information related to health care, such as patient information (e.g., patient demographic information), insurance information, billing data, clinical patient notes, and/or charts, and inventories of supplies. Such a master database can be used in any of a variety of contexts, such as being used for business purposes, to manage financial data, and/or to manage clinical data. The disclosed computer-based technology can incorporate, as part of such a master database, information from other health information systems (e.g., external health information systems) and can also share information contained in the master database with such other health information systems.

In a first general aspect, a computer-implemented method includes receiving, at a computer system implementing a health information system using an underlying customer relationship management (CRM) system, a request for a form related to a particular medical order, the request including a user identifier for a first user requesting the form; identifying, by the computer system, a role of the first user based on the user identifier; identifying a particular workflow from among a plurality of workflows based on the medical order and the role of the first user; determining a current status of the medical order along the particular workflow; selecting, based on, at least, the current status and the particular workflow, a particular portion of client-side code from among a plurality of portions of client-side code, the particular portion of client-side code being configured to be executed or interpreted by a client computing device associated with the first user to implement a portion of the particular workflow on the client computing device; and transmitting, by the computer system, the particular portion of client-side code and other information related to the medical order to the client computing device.

Implementations can optionally include one or more of the following additional features. For example, the method can further include receiving, after transmitting the particular portion of client-side code to the client computing device, an indication that the portion of the workflow was performed on the client computing device; and determining, in response to receiving the indication, a new current status of the particular workflow and the medical order based on the indication. The method can also include causing an updated status to be presented on a display screen of the client computing device based on the new current status. The method can also include identifying, based on the new current status and the particular workflow, a next step for the medical order and a second user to perform the next step, where the second user is different from the first user. The method can also include selecting, based on, at least, the next step and the second user, a second portion of client-side code from among the plurality of portions of client-side code, the second portion of client-side code being configured to be executed or interpreted by a second client computing device associated with the second user to implement a portion of the particular workflow on the second client computing device; and transmitting, by the computer system, the second portion of client-side code and other information related to the medical order to the second client computing device. The particular workflow can include a sequence of steps in a linear order, and the new current status can represent a non-linear variation from the sequence of steps in the linear order. The method can also include causing a second workflow, different from the particular workflow, to be initiated or resumed. The role of the first user can be identified from among the group of roles consisting of scheduler, receptionist, nurse, technologist, doctor, transcriptionist, and biller. The role of doctor can be identified from among the group of roles consisting of radiologist, cardiologist, and neurologist. The underlying CRM system can be a Microsoft CRM system. The particular portion of client-side code can be a JavaScript portion of client-side code. The particular portion of client-side code can be configured, when executed or interpreted by the client computing device associated with the first user, to update a dashboard on a display screen of the client computing device. The updating the dashboard can include updating a list of one or more tasks assigned to the first user. The one or more tasks can be grouped by task type. The particular portion of client-side code can be configured to be associated with a user interface feature that is presented on a display screen of the client computing device, and the particular portion of client-side code can be executed in response to a selection of the user interface feature that is presented on the display screen of the client computing device, and the execution of the particular portion of client-side code can cause one or more actions or operations to occur. The user interface feature can be a selectable button. The particular portion of client-side code may not be included in the underlying CRM system. The method can also include providing integration between the computer system and one or more remote computer systems.

In a second general aspect, a computer-implemented method includes receiving, at a computer system implementing a health information system using an underlying customer relationship management (CRM) system, a request for a form related to a particular medical order, the request including a user identifier for a first user requesting the form; identifying, by the computer system, a role of the first user based on the user identifier; identifying a particular workflow from among a plurality of workflows based on the medical order and the role of the first user; determining a current status of the medical order along the particular workflow; selecting, based on, at least, the current status and the particular workflow, a particular portion of client-side code from among a plurality of portions of client-side code, the particular portion of client-side code being configured to be executed or interpreted by a client computing device associated with the first user to implement a portion of the particular workflow on the client computing device; transmitting, by the computer system, the particular portion of client-side code and other information related to the medical order to the client computing device; receiving, after transmitting the particular portion of client-side code to the client computing device, an indication that the portion of the workflow was performed on the client computing device; determining, in response to receiving the indication, a new current status of the particular workflow and the medical order based on the indication; identifying, based on the new current status and the particular workflow, a next step for the medical order and a second user to perform the next step, wherein the second user is different from the first user; selecting, based on, at least, the next step and the second user, a second portion of client-side code from among the plurality of portions of client-side code, the second portion of client-side code being configured to be executed or interpreted by a second client computing device associated with the second user to implement a portion of the particular workflow on the second client computing device; and transmitting, by the computer system, the second portion of client-side code and other information related to the medical order to the second client computing device.

Implementations can optionally include one or more of the following additional features. The particular workflow includes a sequence of steps in a linear order, and wherein the new current status represents a non-linear variation from the sequence of steps in the linear order.

The details of one or more implementations are depicted in the associated drawings and the description thereof below. Certain implementations may provide one or more advantages. For example, the disclosed computer-based technology can provide a single system through which many or all aspects of a medical practice can be managed, which can create or provide greater data integrity (e.g., less likelihood of inconsistent data across different systems), improved efficiency, and cost savings to medical practices, according to some implementations.

In another example, appropriate portions of executable/interpretable code (e.g., scripts) can be identified and provided to client devices that are being used by users within an organization, which can provide features that are customized to the organization. For instance, executable/interpretable codes can be used to create customized workflows across different users within an organization. Executable/interpretable codes can also be used to build health information systems using existing computer systems that may not be specifically designed for use in health care, such as customer relationship management (CRM) systems. Using existing computer systems can provide benefits, such as cost savings to organizations that may already hold licenses to such systems, and using the techniques and systems described herein to modify the existing systems to provide the health care use functionality described herein can streamline the implementation and improve the compatibility, efficiency, robustness, and functionality of such systems, according to some implementations.

Other features, objects, and advantages of the technology described in this document will be apparent from the description and the drawings, and from the claims.

### BRIEF DESCRIPTION OF THE DRAWINGS

FIG. 1 is a conceptual diagram of an example system that can be used to provide a health information system with workflows.

FIG. 2 is a block diagram of an example system that can provide a health information system.

FIG. 3 is a flowchart of an example technique for implementing a workflow as part of a health information system.

FIG. 4 is a screenshot of an example user interface through which users can log into a health information system.

FIGS. 5-45 are screenshots of example user interfaces for the health information system.

Like reference symbols in the various drawings indicate like elements.

### DETAILED DESCRIPTION

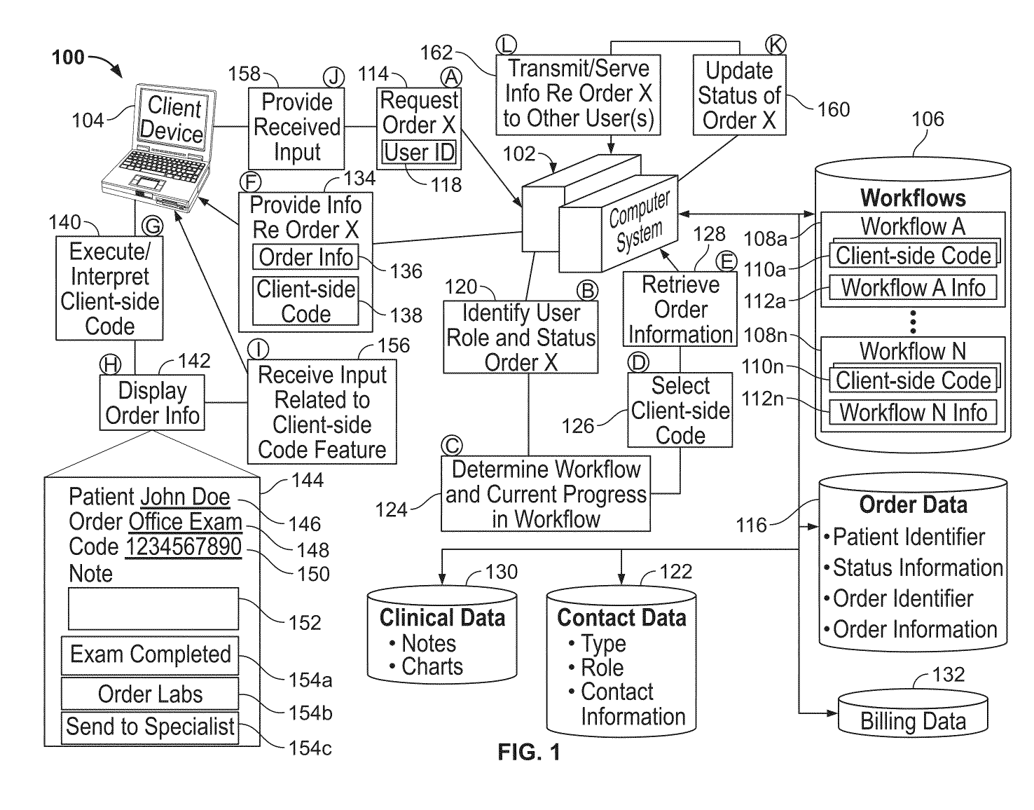

FIG. 1 is a conceptual diagram of an example system 100 that can be used to provide a health information system with workflows. The system 100 includes an example computer system 102 that serves and manages information (e.g., workflow data, patient demographic data, contact data, billing data, or other data) for the health information system, which can be used by users through client computing devices, such as the example client device 104. In particular, the computer system 102 can provide workflows for the health information system that are customized to the health information system and that include order and other workflows across multiple different roles. In general, the systems and techniques described herein may be applicable in any healthcare setting. While the descriptions that follow will in some examples focus on specific roles or healthcare specialties, the systems and techniques may generally be used in any healthcare environment, for example.

For example, a workflow for a patient visit can include tasks performed by a scheduler (e.g., schedule appointment, verify insurance, reminder regarding appointment, schedule follow-up appointments), a receptionist (e.g., check-in patient upon arrival), a nurse (e.g., room patient, take and record patient vital signs, triage patient's reason for visit), a doctor (e.g., perform exam, place any necessary orders, chart patient visit), others fulfilling the doctor's orders (e.g., imaging technician, phlebotomist, specialist), and billers (e.g., verify appropriate billing code, process insurance claim for patient or procedure, follow-up with patient for any outstanding balance). Such a workflow for a patient visit can include sub-workflows that are performed by each of the parties that handle one or more aspects of the patient visit. For instance, there can be a scheduler sub-workflow and a nurse sub-workflow for the patient visit that are performed.

The computer system 102 can include one or more computing devices, such as a computer server system with one or more computer servers, a shared server system, a dedicated server system, a cloud computing system, a desktop computer, a laptop computer, or any combination thereof. The computer system 102 can provide the health information system using one or more underlying technologies, such as a CRM system, and can modify or adapt the underlying technologies for use as a health information system. A CRM system, like MICROSOFT CRM, manages contacts for organizations, such as customers, sales leads, and personnel within an organization. CRM systems may come out of the box with a variety of features, including user interfaces, reporting, and organization-wide accessibility. CRM systems can also include the ability for organizations to customize and adapt the CRM systems for their specific use, such as customizing forms through which data is entered, designating particular fields for contacts, and permissions for users.

The computer system 102 can include a CRM system that has been adapted, through customized entities, user interfaces, executable/interpretable code (e.g., scripts, executable code), and other customizations, to operate as the health information system. Such customization can be extensive, as a CRM system is not configured to manage and track the complexities of a health information system. One area in particular where a CRM system is deficient is with regard to workflows (processes) that are used within the healthcare setting. Workflows are processes for organizations within the healthcare field to use to effectively and efficiently provide medical services to patients. Workflows can be used to determine what tasks should be performed based on a variety of factors (e.g., patient information, current status of order/examination/treatment), when those tasks should be performed (e.g., immediately, within X number of days), who is responsible for performing the tasks (e.g., medical doctor, scheduler, nurse), and/or other details regarding patient care and associated services. Given the wide array of resources (e.g., medical professionals, medical equipment) and the wide array of services offered within the healthcare industry (e.g., primary care examination, specialist examination, imaging, diagnostic information), workflows can be difficult to effectively manage and implement. Typically, each healthcare facility may have a unique set of workflows or processes, or unique steps within their set of workflows or processes, and the customization and integration options provided by the systems and techniques discussed herein can advantageously be used to improve the healthcare experience as compared to the experience provided by traditional CRM systems, in some examples.

CRM systems, however, may not provide features to implement workflows in an intuitive and seamless way so that users within an organization can use them with little, if any, errors. The computer system 102 can build upon a CRM system by including custom client-side code portions (e.g., scripts) that are designed to be executed/interpreted on appropriate client computing devices at appropriate points in workflows so as to provide user interface features and information to the computer system 102 to implement and manage the progress of the workflows. For example, the computer system 102 can provide JavaScript code (example client-side code portion) to the client computing device 104 that is customized to provide user interface features (e.g., selectable buttons) that a user of the client computing device 104 can select to report the user's work on an order and to route the order to an appropriate next step in the workflow. The computer system 102 can determine, based on information that can be received by the computer system 102 from the client computing device 104 through interpretation of the JavaScript code, a next portion of the workflow that should be performed. The computer system 102 can also proceed to effectuate performance of such a determined next portion of a workflow, such as through listing that portion on a dashboard (e.g., list of current work tasks) of a user who has been identified to perform the next portion and/or by transmitting notification of the next portion to the user (e.g., send push notification to the user's device, send email to user).

The computer system 102 can access a repository of workflows 106 (e.g., database, file system, data repository) to manage the flow of work within a healthcare system, such as the information that is provided to particular users, the tasks that those users are asked to perform, the information that is collected through those users (e.g., information collected through forms or by other manners), and who and how other users are engaged to handle other aspects of the workflows. For example, the workflows can be used to determine what forms are presented to users within an organization, what information is auto-populated within these forms, what information is being requested from the user, and what next steps are available for the user to designate. The workflow repository 106 can include multiple different workflows, as indicated by example workflows A-N (108a-n). Each of the example workflows A-N (108a-n) are depicted as each including multiple portions of client-side code 110a-n (e.g., scripts, executable code) that can be executed/interpreted by client computing devices and can also including workflow information 112a-n that identifies information that can be used by the computer system 102 to manage and process workflows, such as information identifying steps and flows between the steps of the workflows, and other appropriate information.

Example steps A-L, which are depicted as being performed across the computer system 102 and the client computing device 104, are an example use of one or more of the workflows 108a-n by the computer system 102 to provide appropriate information, user interfaces, and user interface features on the client computing device 104 to ensure that the appropriate workflow is implemented and followed correctly. The client computing device 104 can be any of a variety of appropriate computing devices, such as a desktop computer, a laptop computer, a mobile computing device (e.g., smartphone, personal digital assistant (PDA), wearable computing device), a tablet computing device, and/or other appropriate computing devices. The client computing device 104 can communicate with the computer system 102 to implement the workflows over one or more appropriate communication networks, such as the Internet, local area networks (LANs), wide area networks (WANs), virtual private networks (VPNs), wireless networks (e.g., WiFi networks, mobile data networks (e.g., 3G/4G networks)), wired networks (e.g., fiber optic networks), or any combination thereof.

As indicated by step A (114), the client computing device 104 can transmit a request for “order X” (X is an example unique identifier for an order) to the computer system 102. Information regarding order X can be stored in an order data repository 116 that is accessible to the computer system 102. The order data repository 116 can store a variety of information regarding orders, such as a unique order identifier, a patient identifier that identifies a patient to whom the order pertains, status information for the order, and other order information (e.g., description of the order, notes regarding the order). The request that is transmitted by the client computing device 104 can be generated in response to user input on the client computing device 104, such as a user selecting an entry for “Order X” from a list of orders that are pending for the user (e.g., list of orders presented on a dashboard for the user). Such a list of orders can have been transmitted to the client computing device 104 before the request for order X is transmitted to the computer system 102. The request can include a user ID 118 for the user of the client computing device 104 (or other information that can be used to identify a person who is using the client computing device 104, such as login credentials, username, and biometric identifiers). In some examples, the user ID 118 identifies the user. In some examples, the user ID 118 identifies a role or position of the user. In some examples, the user ID 118 identifies the client computing device 104, where the client computing device 104 may be associated with the user (e.g., the user logged in or registered at the client computing device 104). In some examples, the user ID 118 identifies two or more of the foregoing.

The computer system 102 can, in response to receiving the request, identify a role of the user of the client computing device 104 and a status of order X, as indicated in step B (120). The computer system 102 can access the order data repository 116 and a contact data repository 122 to identify such information. The contact data repository 122 can store information regarding patients, medical providers (e.g., physician), referring physician, other users within a healthcare organization (e.g., schedulers, billers), and other appropriate users. The contact data repository 122, which can be a contact repository from an underlying system, such as a CRM system, can include a variety of fields/entities that can be customized for use within a health information system, such as fields/entities identifying contact types (e.g., patient, provider, referring physician, organization employee/affiliate), roles within an organization (e.g., medical service provider, scheduler, biller), and/or contact information (e.g., email, phone, address). The user ID 118 can be used to identify information about the user of the client computing device 104, including the user's role within the organization (e.g., physician, nurse, scheduler, technologist, radiologist, transcriptionist).

The user role and status of the order can be used by the computer system 102 to determine which of the workflows A-N (108a-n) to use and the current progress along the applicable workflow, as indicated by step C (124). For example, the computer system 102 can determine that workflow A (110a) applies to order X and that the order X is currently in a third of six steps that are part of workflow A. Such a determination can be based on the status of the order and/or the role of the user. Different workflows and different portions of the workflows can apply based on the role of the user. For example, different portions of workflow A (108a) may apply to (be performed by) a physician and a scheduler, or different workflows may be used for the physician and the scheduler. Workflows may be nested such that they can be readily and easily combined to create larger and more complex workflows. For example, a workflow for a scheduler (e.g., a workflow a scheduler will follow to schedule and confirm patient visits/exams) can be used as a sub-part of other workflows, such as a first workflow in a pediatric clinic and a second workflow in an internal medicine clinic.

The computer system 102 can select one or more appropriate portions of client-side code to provide to the client computing device 104 based, at least in part, on one or more of the workflow that was identified, the progress of the workflow, the role of the user of the computing device 104, information regarding the order (e.g., type of order, status of order), patient information (e.g., gender, age, medical conditions, medical history, previously used medical professionals), and/or other appropriate factors, as indicated by step D (126). For example, if the workflow A (108a) is identified, one or more of the client-side portions of code 110a-n can be selected that are appropriate for a current progress through the workflow A (108a). As discussed above, the client-side portions of code (e.g., client-side code portion 110a) can be executable and/or interpretable by the client computing device 104 and can provide features on the client computing device 104 to aid in the implementation of the workflow A (108a), which, in some implementations, may not be possible with the underlying technology used by the computer system 102, such as a CRM system. The client-side codes (e.g., one or more of codes 110a-n) can collectively, when provided at appropriate points along the workflow A (108a) and to an appropriate computing device, provide features that allow for the workflow A (108a) to be implemented and followed across a plurality of different users within an organization and across a plurality of different client computing devices (e.g., the client computing device 104).

The computer system 102 can also retrieve other information that may be relevant to the order, given the status of the order, the role of the user of the computing device 104, the progress of the workflow, and/or other appropriate information, as indicated by step E (128). Such other information can be retrieved from the order data repository 116, the contact data repository 122, a clinical data repository 130 (e.g., a repository that can store medical history information, notes, charts), a billing data repository 132 (e.g., a repository that can store insurance information, outstanding invoices, history of payments, copay information), and/or other appropriate data sources.

The computer system 102 can provide the information regarding order X to the client computing device 104, as indicated by step F (134). The information can include order information 136 that has been retrieved and the appropriate client-side code 138 that was selected by the computer system 102.

In response to receiving the information from the computer system 102, the client computing device 104 can execute/interpret the client side code 138, as indicated by step G (140). For example, in some implementations the client-side code is a script (e.g., JavaScript) that is interpretable by the client computing device 104 (e.g., interpretable by an application running on the client computing device 104, such as a CRM client application). Interpretation/execution of the client-side code 138 by the client computing device 104 can cause the client computing device 104 to perform any of a variety of operations, such as presenting one or more user interface features (e.g., selectable buttons, voice input prompts, gesture monitoring features) and/or performing operations in response to interaction with such features (e.g., transmitting messages to the computer system 102 based on the user interaction with the interface features).

The client computing device 104 can additionally display (or otherwise present) the order information in association with the execution/interpretation of the client-side code, as indicated by step H (142). An example user interface 144 (e.g., electronic form) is depicted as displaying example order information 146-152 and user interface features 154a-c created through execution/interpretation of the client-side code 138. Some of the order information may not be editable in the interface 144, such as the patient name 146, the order type 148, and a billing/medical code for the order 150. Other fields may be editable, such as the note field 152, into which a user can enter a note regarding the order for the patient.

The user interface features 154a-c in this example are selectable buttons that, when selected, can each perform different operations on the client computing device 104, such as accessing different information on the client computing device 104, transmitting different information to the computer system 102, and/or directing the user interface 144 to a different screen (e.g., interface with additional/follow-up fields). In some examples, the user interface features (e.g., 154a, 154b, and/or 154c, or others) may be associated with client-side code provided by the computer system 102 (e.g., one or more portions of the received client-side code 138), and when the client computing device 104 receives a selection of the user interface feature, the associated client-side code may be executed and may cause one or more actions or operations to occur. In some examples, the one or more actions or operations can affect the order or the workflow, such as causing by an update in a status of the order or of the workflow, or causing a workflow to move to a different stage in the workflow. In some examples, the one or more actions or operations can cause a handoff between actors or users of the system, or can cause a task to become active for another user of the system. In some examples, the one or more actions or operations can cause the current order or the current workflow to be paused or suspended. In some examples, the one or more actions or operations can cause a different order or a different workflow to be initiated or resumed.

Referring again to FIG. 1, for example, a selection of the “exam completed” button 154a can cause information indicating that the exam has been completed to be transmitted from the client computing device 104 to the computer system 102, which may cause the computer system 102, using an appropriate workflow 108a-n, to close out the order and/or transfer the order to another user within the organization, such as a biller. In another example, selection of the “order labs” button 154b can cause the user interface 144 to display additional fields through which the appropriate labs can be selected and can cause the computing device 104 to transmit, to the computer system 102, information adding the labs to the order, which can cause the computer system 102, using an appropriate workflow 108a-n, to transmit the labs order to another user (e.g., lab technician) and to update the status of the order for the patient. In a further example, selection of the “send to specialist” button 154c can cause the user interface 144 to display additional fields through which a specialist (e.g., radiologist, cardiologist, neurologist, or other specialist) can be selected and can cause the computing device 104 to transmit, to the computer system 102, information identifying the specialist referral, which can cause the computer system 102 to, using an appropriate workflow 108a-n, to transmit the specialist request and to update the status of the order for the patient.

As indicated by the buttons 154a-c and the different outcomes that may result by the computer system 102, the workflows may not be linear and may instead branch depending on various factors, such as medical provider judgments (e.g., indicate exam completed, to order labs, or to send to specialist) and/or other factors (e.g., patient medical information and history).

The client computing device 104 can receive input related to the client-side code features, as indicated by step I (156). For example, the client computing device 104 can receive input selecting one or more of the buttons 154a-c. In response to receiving the input, the client computing device 104 may display additional fields and/or forms through the user interface 144, and can additionally provide information regarding the received input to the computer system 102, as indicated by step J (158).

The computer system 102 can receive the input from the client computing device 104 and, based on the input (e.g., user selection of one or more of the buttons 154a-c), can update the status of the order X, as indicated by step K (160). In some examples, the status can be updated based on direction of the client-side code that created the selected feature. For example, client-side code that is executed/interpreted to present the button 154a can include instructions that, when the button 154a is selected, that information identifying an appropriate status update for the order X (e.g., information to update the order to a status of “closed” or “exam completed”). Alternatively and/or additionally, one or more of the workflows 108a-n can be consulted to determine an appropriate status update for the order X based on the user input. The order can be updated in the order data repository 116.

Based on an updated status of an order, the computer system 102 can cause an applicable workflow to progress to a next step in the workflow. In some examples, the next step in the workflow may be applicable to a different user (e.g., a user different than the user associated with client computing device 104). For example, the next step in the workflow may be handled by a different user and/or may be performed as part of a different workflow (e.g., sub-workflow). Using the updated status information, the computer system 102 can transmit and/or serve information regarding the order X to one or more appropriate users who, given their roles within the organization, the status of the order X, and the progress along the applicable workflow, are responsible for handling the next step of the workflow, as indicated by step L (162). For example, the information regarding order X can be provided to another computing device (not shown in FIG. 1) for such another user and can be listed in a dashboard for the other user. That user can then, upon selection of the information for order X, initiate the performance of steps A-L by another computing device associated with the other user and the computer system 102.

In some examples, the data repositories 106, 116, 122, 130, and 132 can be part of a master database of health information that combines information that was formerly distributed across disparate systems. Such collected and available information in a master database, and the systems and techniques discussed herein, can provide a variety of benefits, such as being able to provide more accurate estimates and accounting of the total/true costs of care for various aspects of healthcare services, such as the true cost for patient exams. For instance, the cost for a healthcare organization to provide a patient exam can be based on a variety of information, such as provider fees (e.g., physician fees), facility fees (e.g., building rental/leases, room charges), supplies (e.g., consumables), assets (e.g., reusable medical equipment), and/or other appropriate costs (e.g., support staff, utilities, marketing, billing/accounting costs, collection costs). By having a system and master database that combines all of these information sources, more accurate costs can be estimated and determined, which can improve budgeting, forecasting, streamlining, and overall financial solubility of medical organizations. Additionally, it can allow for medical services to be priced at rates that more accurately reflect the actual costs, which in some instances may increase the prices that patients and insurers pay and in other instances may decrease the prices that patients and insurers pay for medical services. As another example, the system, using information from the master database, for example, can estimate workloads, such as a quantified amount of work to be performed for various aspects of healthcare services. As yet another example, the system, using information from the master database, for example, can estimate production, such as an amount of work scheduled, produced or performed for various aspects of healthcare services.

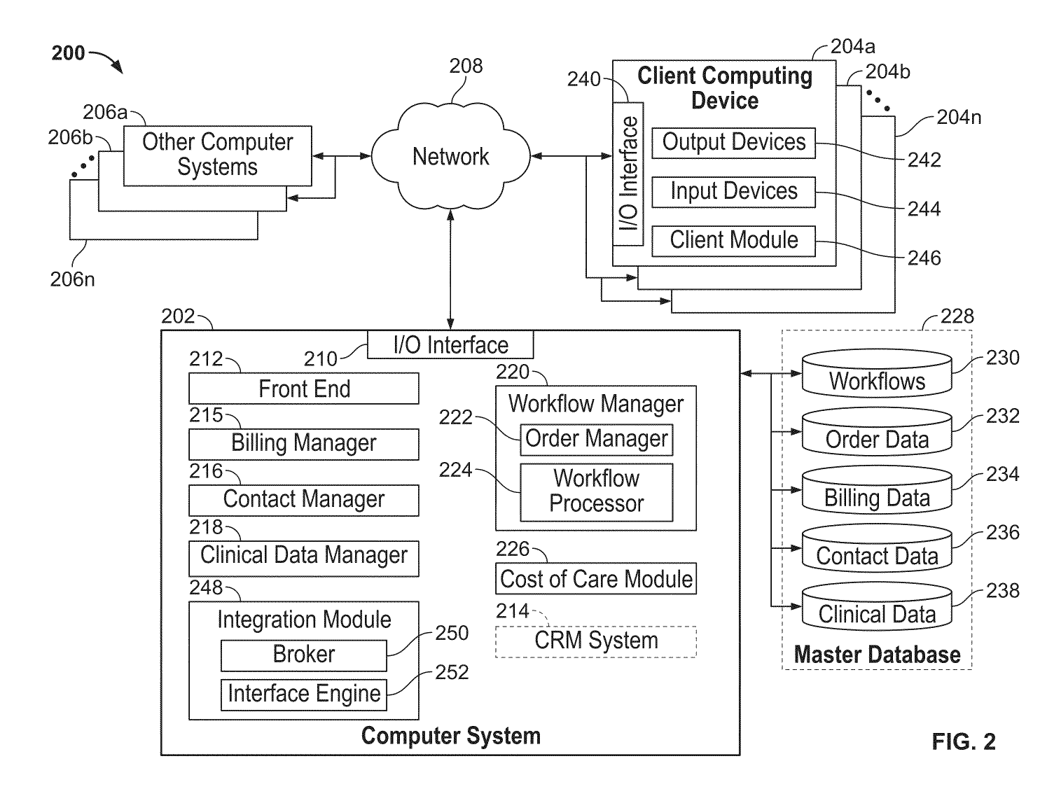

FIG. 2 is a block diagram of an example system 200 that can provide a health information system. The example system 200 can be similar to the example system 100, described above with reference to FIG. 1.

The system 200 includes a computer system 202, client computing devices 204a-n, other computer systems 206a-n, and a network 208. The computer system 202 can be similar to the computer system 102 that is described above, and can be any of a variety of appropriate computer systems, such as a computer server system, a cloud-based computing system, a shared server system, a dedicated server system, and/or a collection of one or more computing devices. The client computing devices 204a-n can be similar to the client computing device 104 described above, and can be any of a variety of appropriate computing devices, such as desktop computers, laptop computers, mobile computing devices, tablet computing devices, wearable computing devices, and/or other appropriate computing devices. The computer systems 206a-n can be any of a variety of external computer systems and/or data sources, such as practice management systems (PM), radiology information systems (RIS), electronic medical record systems (EMR), and/or electronic health record systems (HER). The network 208 can be any of a variety of appropriate communications networks over which the computer system 202, the client computing devices 204a-n, and the other computer systems 206a-n can communicate with each other, such as the Internet, LANs, WANs, VPNs, wireless networks, wired networks, peer to peer networks, or any combination thereof.

The computer system 202 includes an input/output (I/O) interface 210 through which the computer system 202 can communicate over the network 208. The I/O interface 210 can be any of a variety of appropriate interfaces, such as wireless network cards and/or wired network cards. The computer system includes a frontend 212 that the computer system 202 can use to receive, process, and respond to various requests for information from the client computing devices 204a-n and the other computer systems 206a-n. The frontend 212 can be part of one or more underlying technologies that are used by the computer system 202, such as a CRM system 214. The frontend 212 can process and respond to such requests by accessing the other components 215-226 of the computer system 202 and/or a master database 228 that includes data subsystems 230-238.

The other components of the computer system 202 include a billing manager 215 that is programmed to service requests for and manage data within the billing data 234; a contact manager 216 that is programmed to service requests for and manage data within the contact database 236; a clinical data manager 218 that is programmed to service requests for and manage data within the clinical database 238; a workflow manager 220 that is programmed to manage and process the workflows 230 to determine what information should be provided to particular users and for particular orders; and a cost of care module 226 that is programmed to use various analytics and techniques to determine and track supply costs, product pricing and other billing units/data, which the system can use to determine true costs of care for a healthcare organization through use of the master database 228. In various implementations, any of the data or information that is analyzed and tracked by the systems described herein can be included in a wide range of charts or reports. In some examples, the determined cost information can be used with an accounting system, and the systems described herein can be integrated with an accounting system that performs financial accounting functions.

The workflow manager 220 can further include an order manager 222 that is programmed to manage and appropriately update orders within the order database 232 based on information received from the client computing devices 204a-n, such as status updates provided through selectable features (e.g., buttons 154a-c or other buttons or selectable features) implemented on the client computing devices 204a-n through appropriately selected and served client-side codes (e.g., the client side codes 110a-n). The workflow manager 220 can also include a workflow processor 224 that is programmed to process workflows contained within the workflow database 230 in order to determine appropriate information, including appropriate client-side codes, to provide to users depending on a variety of factors, including the users' roles (e.g., physician, scheduler, nurse, technologist, biller, specialist), status of an order, applicable workflow(s) within the workflow database 230, and/or other appropriate factors.

The master database 228 can be local to the computer system 202 (e.g., data storage system accessible via one or more direct physical connections to the computer system 202) and/or remote from the computer system 202 (e.g., remote data storage system, such as a cloud-based data storage system). In some examples, the master database 228 may be housed within a single storage device or component, and in some examples the master database 228 may be distributed across multiple storage devices or components.

The client computing devices 204a-n can each include an I/O interface 240 through which the client computing devices 204a-n can communicate over the network 208, one or more output devices 242 (e.g., display, speakers), one or more input devices 244 (e.g., keyboard, mouse, track pad, buttons, touchscreen, microphone, camera), and a client module 246 that is programmed to present information received from the computer system 202 using the output devices 242, to receive user input, and to provide appropriate information to the computer system 202 based on the user input as part of a client device in a health information system. The client module 246 can be a standalone application (e.g., application running on WINDOWS OS, application running on MACOS, mobile device application) that is installed on the client computing device 204a. The client module 246 can also be a web-based application that is loaded from the computer system 202 and run on the client computing device 204a through a web browser application installed on the device 204a. The client module 246 is programmed to execute/interpret the client-side code portions (e.g., the client-side code 110a-n).

The computer system 202 can also include an integration module 248 that is programmed to map data from other computer systems 206a-n to the master database 228, and vice versa, and to manage the interactions between the computer system 202 and the other computer systems 206a-n to facilitate the sharing of data. The integration module 248 includes a broker 250 and an interface engine 252. The broker 259 can translate data from one language to another. The interface engine 252 can map the fields used by a first system to the fields used by a second system. The integration module 248 can be used as a one-to-one integrator, such as from a first or host system to a second system, vice versa, or bidirectionally between the two systems. The integration module 248 can be used as a one-to-many integrator, such as from a first or host system to two or more other systems, from two or more other systems to the first or host system, or bidirectionally/multidirectionally between the first or host system and the two or more other systems. The integration module 248 can be used as a many-to-many integrator, such as from two or more systems including a first or host system to two or more other systems, from two or more other systems to two or more systems including a first or host system, or bidirectionally/multidirectionally between the various systems. For example, integration can be performed in bi-directional or multi-directional flows. The integration module 248 can use one or more mappings of data fields to perform such data sharing, in some examples.

The components 212-226 and 248-252 of the computer system 202 and the client module 246 of the client computing device 204a can be implemented in any of a variety of ways, such as through software (e.g., executable/interpretable instructions), hardware (e.g., application specific integrated circuits (ASICs), one or more general purpose processors programmed to perform particular operations), firmware, or any combination thereof.

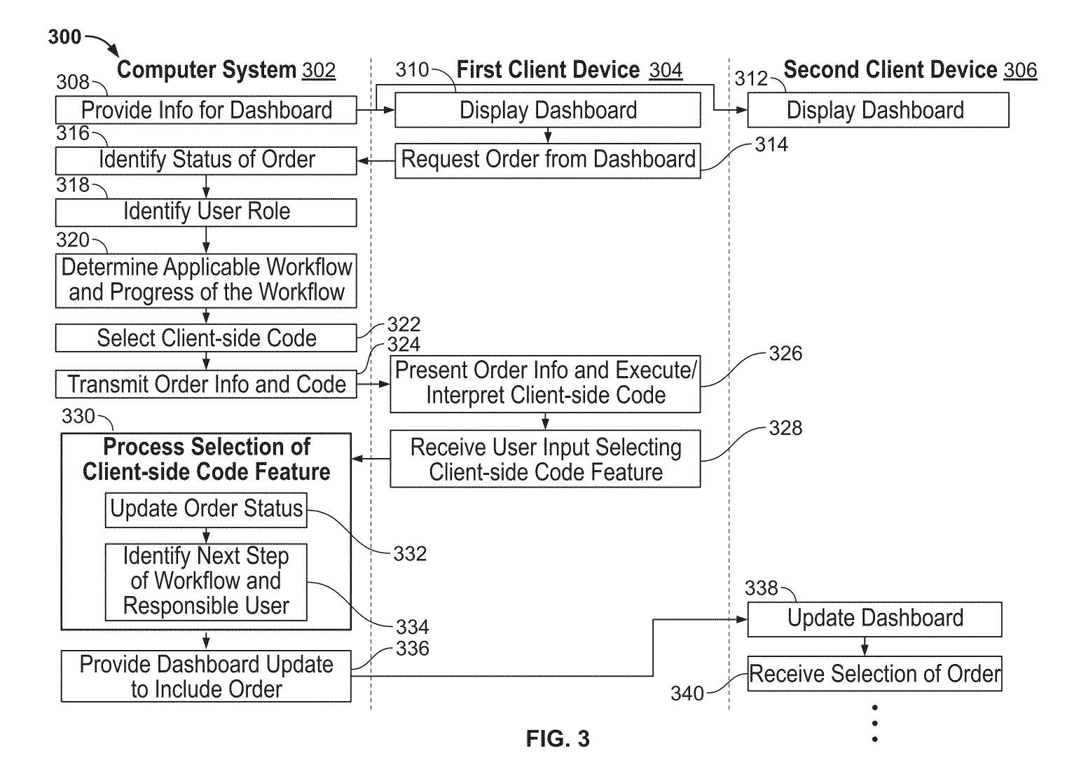

FIG. 3 is a flowchart of an example technique 300 for implementing a workflow as part of a health information system. The example technique 300 can be performed in part by an example computer system 302, an example first client computing device 304, and an example second client computing device 306. The computer system 302 can be any of a variety of appropriate computer systems, such as the computer system 102 (see FIG. 1), the computer system 202 (see FIG. 2), and/or other appropriate computer systems. The first client computing device 304 and the second client computing device 306 can be any of a variety of appropriate client computing devices, such as the client computing device 104 (see FIG. 1), the client computing devices 204a-n (see FIG. 2), and/or other appropriate computing devices.

The computer system 302 can provide information for one or more dashboards to be displayed to users of the first client computing device 304 and the second client computing device 306 (308). A dashboard can include one or more lists of work tasks that are assigned to a user, and they may be grouped according to the task type. For example, a physician may have a dashboard that includes a first list of upcoming patient visits for the day, a second list of lab results that need to be analyzed, and a third list of previous patient visits for which a chart has not yet been completed. Different information can be provided to the first client computing device 304 and the second client computing device 306, and the information that is provided can be based on the role of the associated users (e.g., doctor, nurse, scheduler, technician) of the respective client computing devices. In some examples, the same information is provided to both client computing devices 304, 306, but only a subset of the provided information may be usable or accessible by a particular client computing device 304 or 306 based on a privilege or access parameter associated with the client computing device, for example based on a role of the user of the device.

The first client computing device 304 that is associated with a first user and the second client computing device 306 that is associated with a second user can display the information on dashboards (310, 312). The first client computing device 304 can receive input selecting an order (e.g., patient exam, pending labs) from the dashboard and can transmit a request for the order to the computer system 302 (314).

The computer system 302 can receive the order request and can proceed to identify and retrieve relevant information related to the request, such as appropriate information about the order given the order's current status and the progress along an applicable workflow. The computer system can also identify and/or select appropriate user interface controls to be provided on the first client computing device 304 to ensure that the order progresses to an appropriate next step along the workflow. For example, the computer system 302 can identify a current status of the order (316), identify a role for the user requesting the order (318), and determine an applicable workflow and the current progress along the workflow (320). Based on one or more of these factors, the computer system 302 can select one or more appropriate portions of client-side code, which can provide controls on the first client computing device 304 that will route the order along the workflow to an appropriate next step (322). The computer system 302 can transmit the applicable order information and client-side code to the first client computing device 304 (324).

The first client computing device 304 can present the order information and execute/interpret the client-side code, which can present one or more client side features, such as user interface controls (e.g., selectable elements) (326). In some examples, the client side features may be associated with portions of client-side code, such that when the client side feature is selected, the associated client-side code is executed. The first client computing device 304 can receive user input selecting one or more of the client-side code features (e.g., user interface controls associated with one or more appropriate client-side code portions) (328), which can be used to implement workflows across a plurality of different devices and a plurality of different users through an underlying computer system (e.g., CRM system) and client modules (e.g., CRM client applications) which are not adapted for intuitive workflow management. The first client computing device 304 can transmit, based on the execution/interpretation of the client-side code, information related to the selection to the computer system 302.

The computer system 302 can receive the information and process the selection of the client-side code feature (330). Such processing can include, for example, updating the status for an order to which the selection pertained (332) and identifying next step(s) of the workflow that are to be performed and/or user(s) who are responsible for handling the next step(s) (334).

In this example, a second user who is associated with the second computing device 306 is identified as the responsible user for performing the next step in the workflow, for example based on the selection of the client-side code feature by the first user associated with the first client computing device 304. Based on the identification of the second user as the responsible party for the next step, the computer system 302 can cause the order to be listed in the second user's dashboard. For example, the computer system 302 can provide an update to the second client device 306 that includes an update to the second user's dashboard to include the order (336).

The second computing device 306 that is associated with the second user can receive and update the display of the dashboard to include the order (338). With the order now presented on the second user's dashboard (order may not have been on the second user's dashboard previously), the second user can now select the order (340). Selection of the order by the second user can cause the second client device 306 and the computer system 302 to perform operations similar to the operations 314-336, as described between the first client device 304 and the computer system 302. Involvement of other users and computing devices beyond the first and second computing devices 304 and 306 are possible, and depend on how the workflow that is being followed by the computer system 302 has been designated. The operations in technique 300, or portions thereof, can be repeatedly performed until an entire workflow has been completed.

The disclosed health information systems can be focused on delivering healthcare business applications that provide experiences that are modern, seamless, and device optimized. Such health information systems can provide a user interface that is cleaner, faster, more intuitive, and that drives productivity; use agile process guidance to let users respond rapidly to changing business needs; obtain access on the go so that users access what they need when they need it; enterprise collaboration so that users can work across traditional boundaries between systems to obtain an improved customer experience; and/or platform enhancements to provide robust platform capabilities for a wide array of solutions.

The disclosed health information systems can be implemented in any of a variety of ways, such as through hosted installations and on-premises installations. Hosted installations can reduce the system requirements of traditional on-premises deployments by operating all the infrastructure and platform essentials in the cloud, according to some examples. An example of minimum requirements for such a hosted installation can include: a WINDOWS operating system when using CRM for OUTLOOK or APPLE MAC when running APPLE SAFARI, supported tablets, and/or mobile devices; supported web browser, such as later versions of INTERNET EXPLORER or the latest versions of APPLE SAFARI, GOOGLE CHROME and/or MOZILLA FIREFOX; and optionally MICROSOFT OUTLOOK OFFICE.

On-premises installations can have a much larger requirement for both hardware and software, in some examples. For example, on-premises versions can require the software listed for the hosted installation plus: a MICROSOFT WINDOWS SERVER, a MICROSOFT WINDOWS SERVER ACTIVE DIRECTORY infrastructure, an INTERNET INFORMATION SERVICES (IIS) website, a MICROSOFT SQL SERVER 2008 or MICROSOFT SQL SERVER 2012, a MICROSOFT SQL SERVER 2008 REPORTING SERVICES or MICROSOFT SQL SERVER 2012 REPORTING SERVICES, a MICROSOFT EXCHANGE SERVER or access to a POP3-compliant email server (optional), a SHAREPOINT SERVER (used for document management), and claims-based security token service (used for Internet-facing deployments).

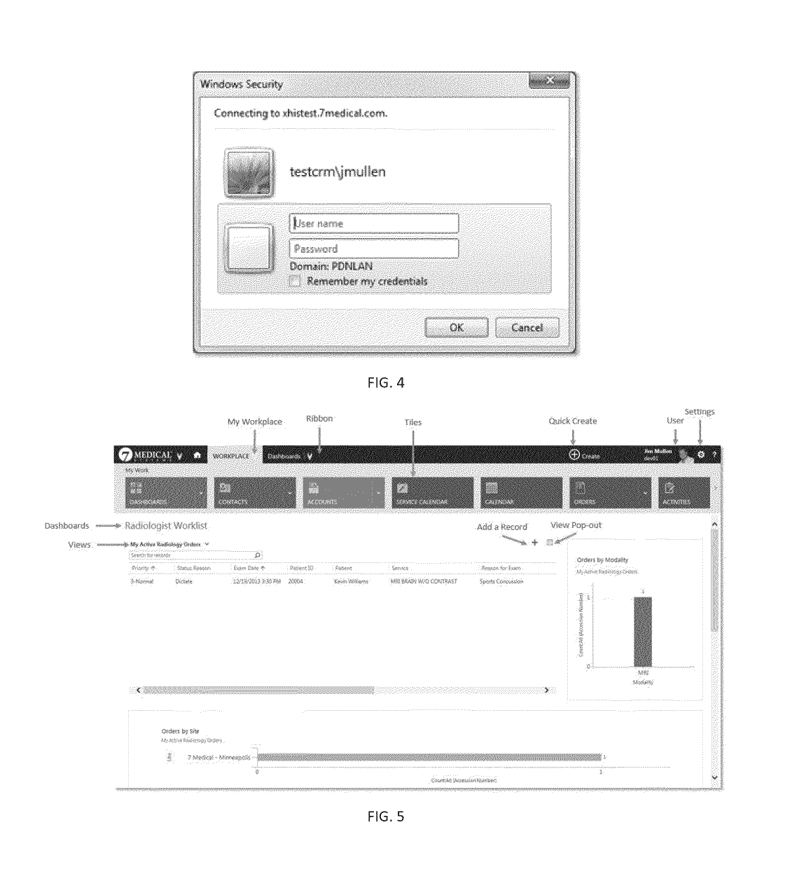

FIG. 4 is a screenshot of an example user interface through which a user can log into a health information system provided by a computer system (e.g., the computer systems 102, 202, and 302).

Logging in to a health information system involves a user entering his/her user ID (user identification) and a password on the login window, as depicted in FIG. 4. A user's user ID can be the name the user enters when logging in to the system (e.g., the computer systems 102, 202, 302). A user's password can be a private code that gives the user access to the system. The user's user ID can be supplied to by a user's organization. A user's initial system password can be the user's user ID. After logging in, the user can be prompted to change his/her system and signature (if applicable) passwords.

FIGS. 5-28 are screenshots of example user interfaces for the health information system. The user interfaces depicted in FIGS. 5-28, either in whole or in part, can be served by computer systems hosting a health information system, such as the computer systems 102, 202, and/or 302 described above, and can be displayed on client computing devices, such as the client computing devices 104, 204a-n, and/or 304-306.

FIG. 5 depicts an example user interface with navigation features for accessing different features within the health information system. The example user interface depicted in FIG. 5 can allow users to view and access a wide array of relevant information in one spot without pop-ups or having to switch from one application to another. For instance, FIG. 5 depicts an example dashboard for a radiologist. A form can be opened from the dashboard by selecting a record/order from the worklist (e.g., “Radiologist Worklist”).

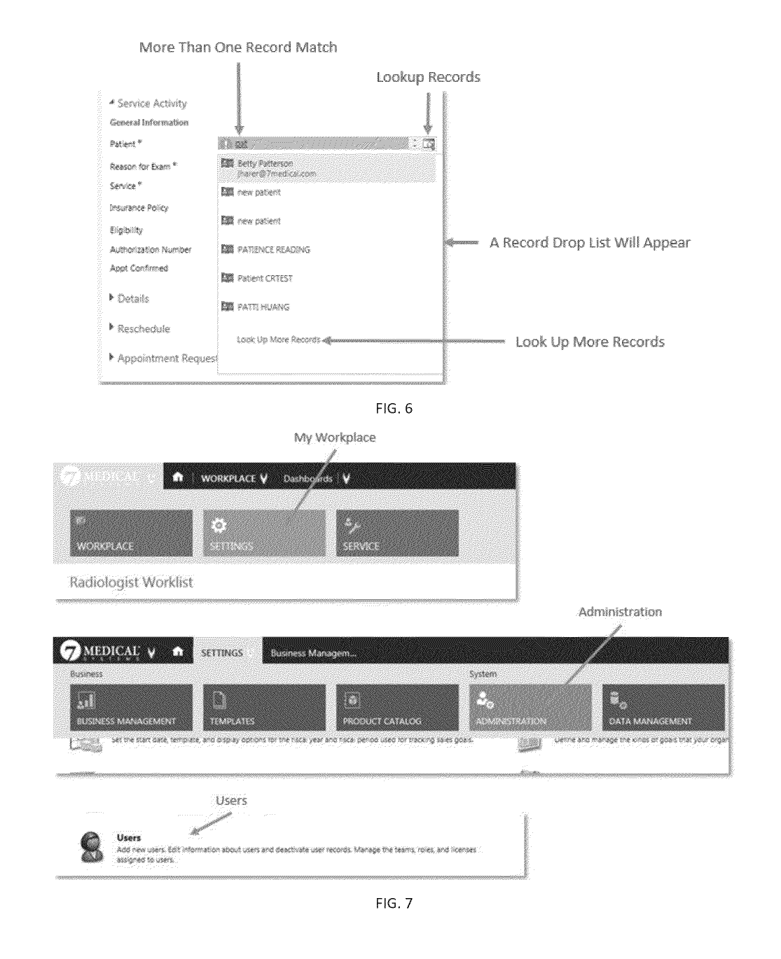

Once a new form has been opened, a user can review, edit, and/or complete information within the form. In the example form depicted in FIG. 6, fields include those listed in the column on the left of the form: general information, patient, reason for exam, service, insurance policy, eligibility, authorization number, and appt confirmed. Required fields can be identified with an asterisk. A user can type into the fields based on the form and, if there is only one matching record, the system may recognize and auto-populate. If there is more than one record available, a user can receive the drop list record indicator depicted in FIG. 6 with the option to look up additional records. A user can click on the magnify glass to lookup a record directly.

Referring back to FIG. 5, several user interface features are identified with annotations to the depicted screenshot. Under the Ribbon Navigation feature, there is a My Workplace feature through which a user can access system and/or individual entities used on a frequent (e.g., daily) basis, and a Settings feature through which a user can access business, system, and/or customization settings, and can access and implement processes. Listed under the “My Workplace” header and identified by the arrow “Tiles,” which can be associated with a workplace entity, are Dashboards (which can be customized), Contacts, Accounts, Service Calendar, Calendar, Orders, and Activities.

Dashboards can act as a business intelligence tool and can provide a snapshot of data in various forms. For example, a user can use a dashboard to simultaneously present data from charts, grids, IFRAMES, and Web resources. A dashboard can act as a container for such objects, and can simultaneously present data from up to a number (e.g., six) of visualizations, grids, IFRAMES, or web resources.

Contacts can be a person who represents a patient and/or potential patient, and/or an individual related to an account. For example, an individual who is being seen for a service or an employee of an account can be a contact. A contact may also be a person involved in a business transaction, such as a supplier or a colleague.

Accounts can include people and/or businesses to which a product or service is related, such as a company that is billed in business transactions. For example, a referring physician group or insurance company can be an account.

Service Calendar (service activity) can include schedulable appointments to provide service to patients. A service activity can use one or more resources (e.g., modalities, rooms, physicians) to perform a service, for example, at a specific time and place.

A Calendar/Appointment can be an activity represented by a time interval that has a start time, an end time, and a duration. An appointment may not include a service or check for conflicts, and may not include a search for available times.

Orders can be confirmed requests for a good or service based on specified terms.

Activities can be actions to be performed, such as tasks, and/or communication items that are sent and/or received, for example, email, phone calls, and appointments. The status of activities can be tracked and the activity history can be stored in the system (e.g., computer systems 102, 202, and/or 302), so that users can view the open and closed activities.

A user can designate his/her user preferences through the interface depicted in FIG. 5, for example, by selecting the ‘7 Medical’ entity to cause a dropdown menu to appear and then, as depicted in FIG. 7, selecting ‘Settings’; then on the ‘Settings’ entity selecting ‘Administration’; and then on the ‘Administration’ entity selecting ‘Users’. On this screen a user can change the following information: General Settings (e.g., Account, User Information, Address Information), Configuration Settings (e.g., Organization, Email Access, CAL Information), Preferences (e.g., Report Delivery, Alerts), and/or PACS Configuration (e.g., PACS Viewer Launch capabilities).

Health information systems can use statuses to represent the state of a record and/or activity. For example, an order can be Active or Resolved, and an email activity can have a status of Draft or Sent. Status can also be used by workflow rules to determine when to move to the next stage in a workflow or sales process. For instance, in one example implementation the status of orders includes Active, Cancelled, and Fulfilled.

An Order Status Reason can be a description of the status of a record or activity. For example, if an order has a status of Active, the status reason could be ‘Begin Procedure’ or ‘Transcribe.’ Example Order Status Reasons include: Requested, Scheduled, Begin Procedure, In Progress, On Hold, Dictate, Transcribe, Proofread, Complete, Canceled, and Amended. In general, these are example statuses applicable to a particular order, or to a particular health information setting. In other examples, alternative statuses may be used as appropriate. In general, the system (e.g., computer system 102, 202, or 302) may use the status as a factor in determining a progress of a workflow, or determining to activate or handoff an order from a first user to a second user, for example.

Common Tasks can be certain tasks and/or functions that are repeatedly performed throughout the disclosed health information systems. Although such tasks and/or functions can be performed at various times, the procedures for completing them may be the same.

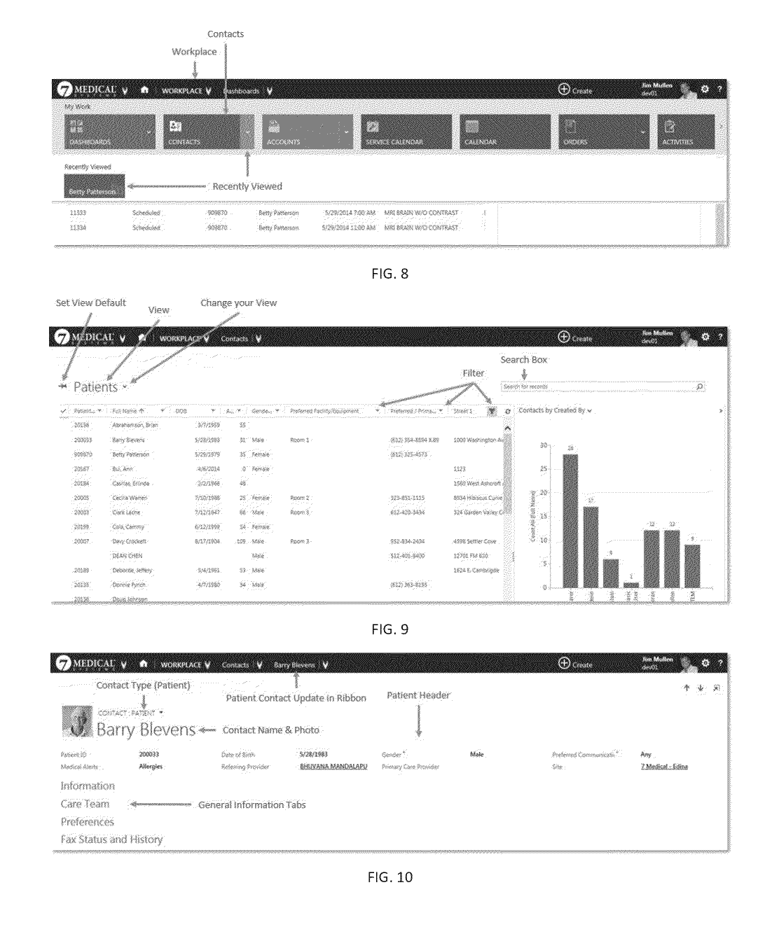

In an example common task to lookup a patient (example Contact), a user can, as depicted in the example user interface in FIG. 8, select ‘WORKPLACE’ and ‘CONTACTS’ from the home screen/dashboard. If the user has recently viewed the patient, the user may select the drop down from the Contacts entity. Referring to the example user interface depicted in FIG. 9, a user's view can default to ‘Patients’, from which a user can search and/or filter based on columns. A Search Box is depicted in the upper right corner. To change the View, a user can select the drop down next to the name of the current view (“Patients” in this example).

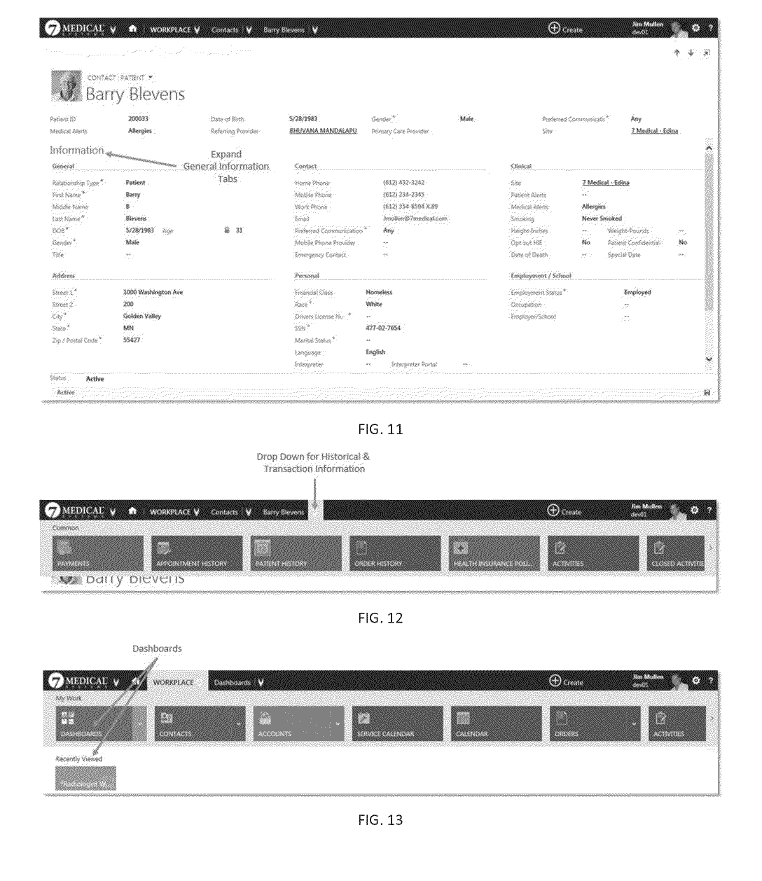

A user can view a patient's contact information using the Contact (Patient) entity by selecting a Contact (Patient) either from the View in FIG. 9 or from the dashboard. A user can use the example user interface depicted in FIG. 10 (which depicts an example contact (patient) entity) to review, edit, and/or update Information, Care Team, Preferences and Fax Status & History. Referring to FIG. 11 (which depicts an expanded view of the user interface depicted in FIG. 10), a user can select Each General Information Tab to expand/collapse the information for review and edit. Referring to FIG. 12 (which depicts an example user interface), to review historical and transaction data about the contact (patient), a user can select the drop down next to the contact name in the patient ribbon to be presented with options including: PAYMENTS, APPOINTMENT HISTORY, PATIENT HISTORY, ORDER HISTORY, INSURANCE, and OPEN AND CLOSED ACTIVITIES.

A Scheduling Dashboard can be used for scheduling a service activity, which can include scheduling and managing exams (e.g., Orders) and exam resources (e.g., Room, Modality, User) at an organization. Such a Scheduling Dashboard can allow a user to perform the following: ordering exams (e.g., entering order requests), scheduling ordered exams, scheduling and rescheduling exams, editing exam data, scheduling walk-ins, canceling exams, opening and closing resources, and/or viewing resource schedules. The Scheduling Dashboard can allow a user to order an exam from within a health information system. If a user's organization normally uses another system to schedule, the user interface can be used to bring the externally scheduled orders directly into the health information system for more detailed and specialized workflows.

Referring to FIG. 13 (example screenshot), a user can access the Scheduling Dashboard by selecting WORKPLACE and then selecting DASHBOARDS. If the user is a scheduler, the user may choose to select this dashboard as his/her default dashboard, for example. A user can also access this through his/her recently viewed dashboards. The Scheduling Dashboard can present the following default views: Today's Active Exams (e.g., exams that are scheduled, requested, or checked-in for that day), All Active Exams (e.g., exams that are scheduled, requested, or checked-in), Unverified Patients (e.g., exams that require insurance eligibility check), Front Desk Team Member's Active Activities (e.g., shared activities that the front desk clerks are responsible for), and/or Patients (e.g., all contact's patients that exist within the system).

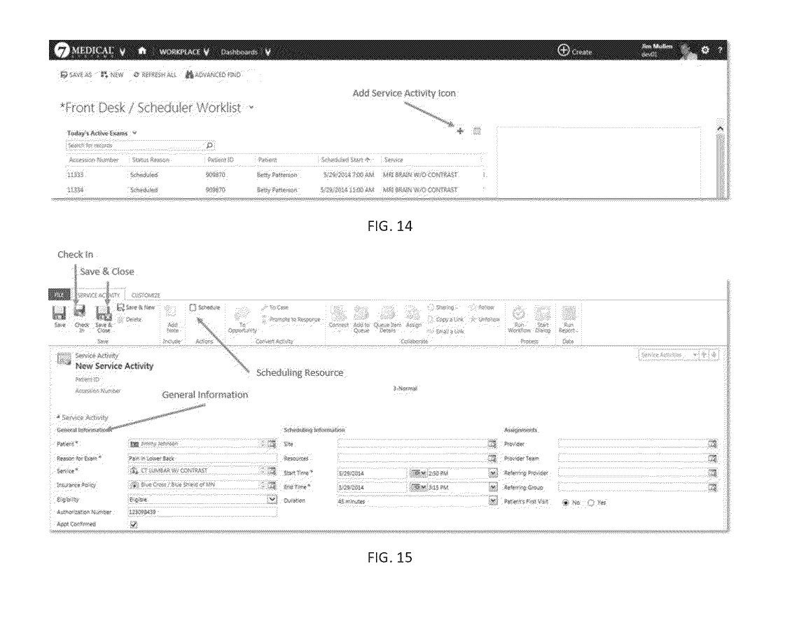

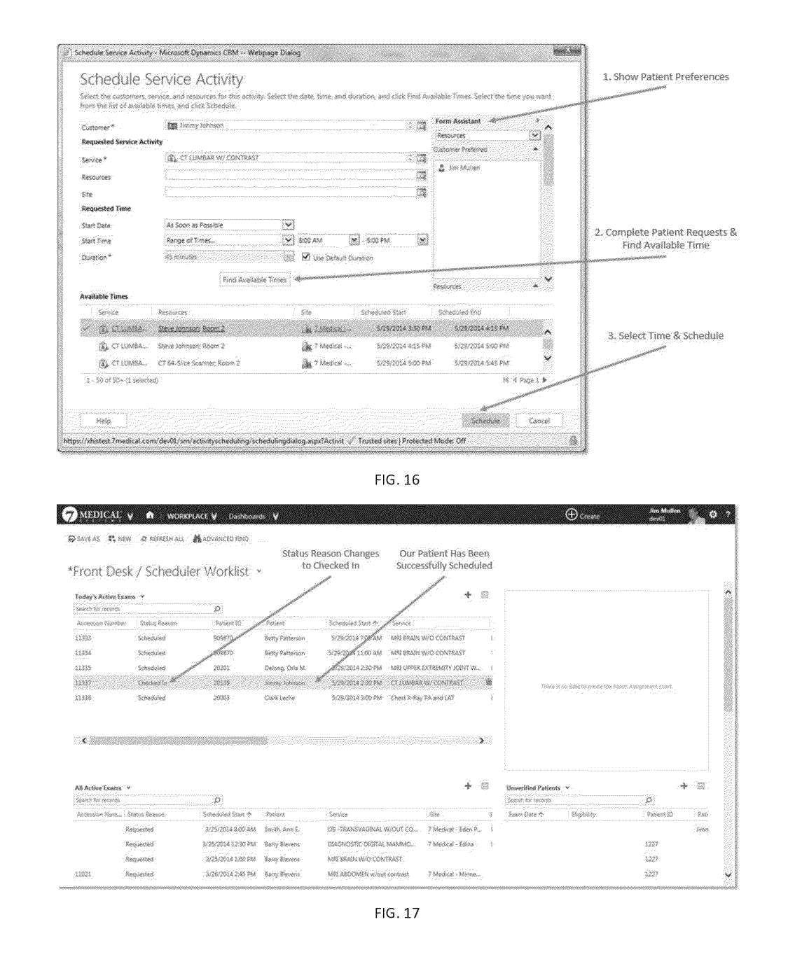

Referring to FIG. 14 (example screenshot), a user can schedule a service activity and/or order (e.g., Exam) by selecting the ‘Add Service Activity’ icon on the Scheduling Dashboard. Then, referring to FIG. 15 (example screenshot), a user can complete the ‘New Service Activity’ Form with the General Information and Scheduling Information. To complete the Scheduling Information, a user can use the Scheduling Resource by selecting ‘Schedule’ from the ribbon area of the screen. A Schedule Service Activity screen will then appear, as depicted in FIG. 16, and a user can select the patient, service and resources for this activity, as well as the date, time, and duration, and then click ‘Find Available Times.’ The user can select a desired time from the list of available times, and then click ‘Schedule.’ A user can use the Form Assistant to view patient specific details on preferred providers, resources, locations and services. The Scheduled Information will fill in on the completed Service Activity, and then a user can proceed with the ‘Save & Close’ or ‘Check-In’ features depicted in FIG. 15. Upon selecting the ‘Save & Close’ feature, the user interface can return to the Scheduling Dashboard. A user can then proceed to Patient Check In if they have not already and the Status Reason can be changed from ‘Scheduled’ to ‘Checked In,’ as depicted in FIG. 17 (example screenshot). For example, the Status Reason may automatically be updated by the system, as by custom script/code that can be executed when the Check In button is selected.

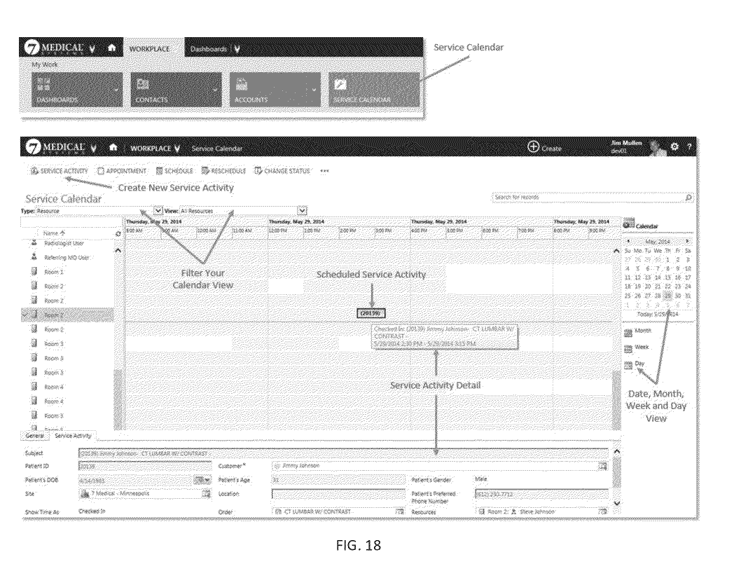

Referring to FIG. 18 (example screenshot), a user can access the Service Calendar and schedule a Service Activity & Order (Exam) From the Service Calendar by selecting WORKPLACE from the Ribbon, selecting ‘Service Activity’, and then repeating the process discussed above with reference to FIGS. 14-17.

Although not depicted in explicit screenshots, the user interfaces depicted in FIGS. 4-18 can be used to access and use other features within a health information system, such as cancelling a service appointment (exam), ordering an exam, accessing an order entry queue, scheduling exams, rescheduling exams, and/or canceling exams. Additionally, health information systems can be used to provide document management solutions, such as storing, modifying, providing access to, and tracking changes to documents that are used within healthcare systems.

The disclosed computer systems can include one or more software applications that are used as the platform for building any health information system. Such health information systems can use the MICROSOFT DYNAMICS CRM application as the underlying system, and can add custom developed code and scripts designed specifically for healthcare.

The disclosed computer systems can be used to build any health information system and can be customizable for any healthcare specialty or application. For example, the disclosed computer systems can be used in specialties such as imaging centers, radiology group practices and other ambulatory clinics, and the disclosed computer systems can be used for business, financial and clinical applications such as practice management (PM), radiology information systems (RIS), electronic medical or health records (EMR/EHR), or supplies and/or asset management systems.

The disclosed computer systems can also serve as a master database that collects patient demographic information; insurance information and billing data; clinical patient notes and charts; and other healthcare related information. As the master database for any business, financial and clinical data, the disclosed computer systems can integrate or send this data to any other health information system, or can receive data from other health information systems, as needed. The disclosed computer systems can support integrations to exchange data from one system to another system (one-to-one), from one system to multiple other systems (one-to-many), or from several systems to multiple other systems (many-to-many). Additionally, the disclosed computer systems can include the following features, which can be used across any healthcare setting: serving as a master patient database that can be integrated to any other health information system; database for collecting patient demographic and health insurance data (PM); database for radiology data (RIS); database for clinical patient data (EMR/EHR); managing a healthcare entity's order workflows (processes); providing portals for patients, referring physicians and attorneys; health information system built on the MICROSOFT CRM platform that will be meaningful-use certified under the HITECH Act; and manage business process flow for SNOMED.

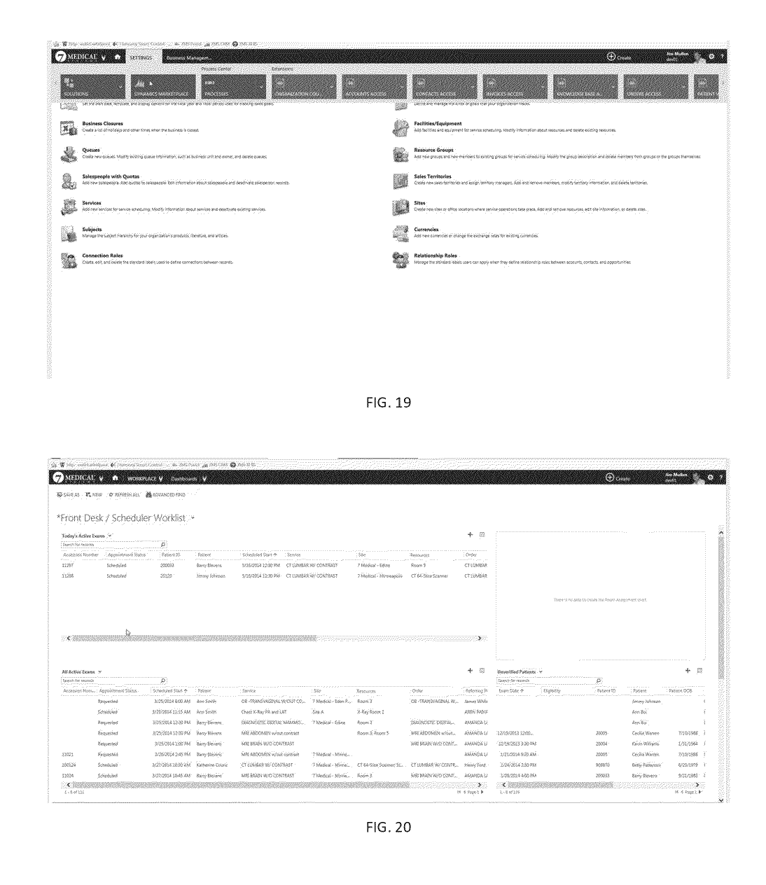

Referring to FIG. 19 (example screenshot), a graphical user interface (GUI) can be used that incorporates at least a portion of existing MICROSOFT CRM user interface features and functionality, and that adds additional customization designed specifically for healthcare settings (e.g., imaging centers, radiology group practices, and other ambulatory clinics). For example, the disclosed computer systems can use custom entities beyond the standard or default entities that are installed with the default installation of MICROSOFT CRM. For instance, the GUI depicted in FIG. 19 displays example standard and custom entities.

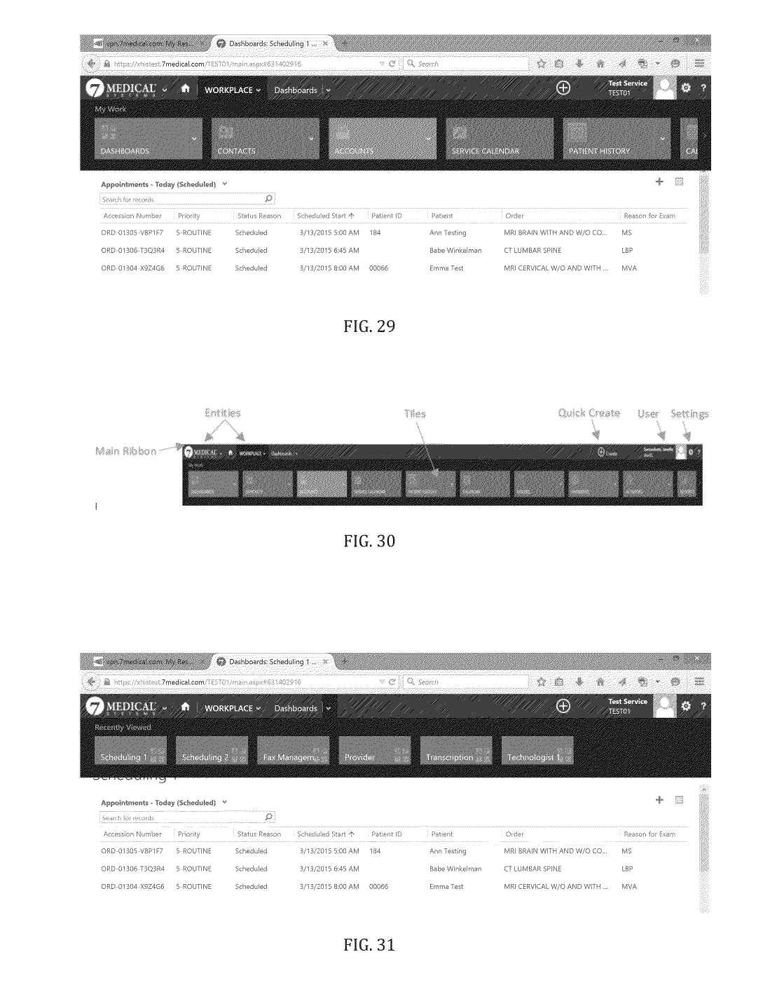

For example, the ribbon feature depicted in FIG. 19 provides navigation functionality. MICROSOFT CRM has a standard ribbon area of the interface that displays navigation buttons horizontally across the top of the screen. The ribbon can be configured to display frequently-used or desired entities. MICROSOFT CRM includes standard entities, and the computer systems discussed herein add a plurality of custom entities (e.g., 46 custom entities) designed specifically for use in healthcare settings or applications to the standard entities. Custom entities, in this example, can be displayed in a different color (e.g., green/teal). FIGS. 29 and 30 are screen shots of example user interfaces that display a workplace ribbon with standard and custom entities. In some examples, the custom entities added by the computer systems discussed herein include Alert, Asset, Attachment, CDR Mapping, CDRI, CDRO, Counter, Diagnosis Code (ICD-10), Diagnosis Code (ICD-9), EDI Destination, Ethnicity, Financial Class Type, Health Insurance Policy, Image File, Language, LOINC, Message, Modifier/Q Code, Order Diagnosis Code, Order Modifier, PACS Variables, Patient History, Payment, Payment Type, Planner Configuration (APEX), Planner Log (APEX), Problem, Profile Album, Quick Text, Race, Race and Ethnicity, Report Images, Report Template, Report Template Related Service, Room, Routing Rule, Service Diagnostic Code (ICD-10), Service Diagnostic Code (ICD-9), Service Product, Service Setting, Site Logo, Site Settings, Smoking Status, SMS Web Service, Time Clock, and Vital Signs.

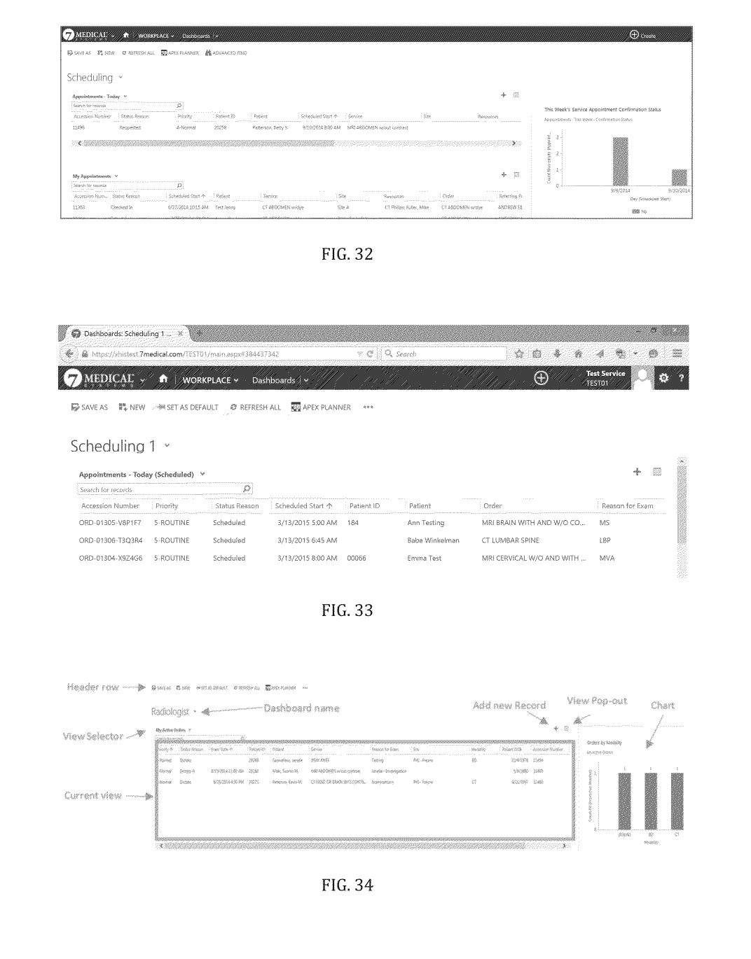

Dashboards can be configured to display useful information for a particular user's role or position. For example, a dashboard can be configured to display a worklist (e.g., activities) and information relevant to the user's role, such as the example Front Desk/Scheduler Worklist (dashboard view) depicted in FIG. 20 (example screenshot). In some examples, a Dashboard is a user interface feature that displays views of worklists and data. For instance, Dashboards can be configured to display views of relevant worklists (activities) and information relevant to respective user roles. Each user role can have a dashboard view configured to prioritize the information relevant to that role. Dashboards can serve as the “homepage” for each type of role. FIGS. 31-34 are screen shots of example user interfaces that provide examples of various dashboards. The user interface of FIG. 31 shows a dashboard with a drop menu, where dashboard views may be made available based on a role or security role of a user, for example. The user interfaces of FIGS. 32 and 33 show example dashboards for a scheduler or front desk receptionist role. The dashboard views include lists of appointments. The user interface of FIG. 33 includes a list of appointments scheduled for today, for example, where each of the appointments includes a Status Reason of “Scheduled”. In some examples, the scheduler dashboard can provide the following information: a list of appointments scheduled, requested, or checked-in for the current day; a list of exams assigned to the user, which in some examples may be labeled “My Appointments;” a list of shared activities assigned to a team (e.g., a front desk team), where an individual activity may be performed by a member of the team; a list of all contacts, or all contacts of a particular type (e.g., patients) within the system. The user interface of FIG. 34 shows an example dashboard for a specialist physician role, a radiologist in the depicted example.

As described previously, the techniques and systems described herein may be applicable to any healthcare setting. The examples that follow will be directed to describing, without limitation, workflows in an imaging center. The user roles in the simplified example described below for the imaging center include a front desk user or scheduler, a technologist, a radiologist, and a transcriptionist. Example roles and role descriptions for users in an imaging healthcare center are listed in TABLE 1 below. Other roles and role descriptions can also be used.

TABLE 1

| Role Title | Role Type | Role Description |
|---|---|---|
| Front Desk or | Administrative | Schedules appointments for patient |
| Scheduler | Staff | exams (orders). |
| | | Collects patient demographic and |
| | | insurance information and enters |
| | | this information into the system. |
| | | Verifies patient insurance eligibility. |
| | | Verifies patient pre-authorization |
| | | for selected services, if applicable. |
| | | Sends patient appointment |
| | | reminders (may be automatic, |
| | | depending on the feature). |
| | | Checks-in patient for exam (order) |
| | | Collects patient payment |
| | | information and payment (i.e., co- |
| | | pays or out of pocket expenses not |
| | | covered by insurance). |
| | | Checks-out patient after exam |
| | | (order) is completed. |
| | | Schedules follow up exam/visit |
| | | (order), if applicable. |
| Technologist | Clinical Staff | Enter or pulls up patient current |
| | | exam (order) and prior exam |
| | | (order) information |
| | | Begins procedure or performs |
| | | patient imaging exam (fulfills order) |
| | | Sends/routes imaging exam (order) |
| | | to radiologist for diagnostic |
| | | interpretation |
| Radiologist | Clinical Staff | Interprets or “reads” the imaging |
| | | exam |
| | | Diagnoses patient condition |
| | | Dictates diagnostic results into |
| | | voice recognition or audio |
| | | recording system |
| | | Sends/routes diagnostic results |
| | | report back to referring physician |
| Transcriptionist | Administrative | Transcribes (types) audio recording |
| | Staff | of radiologist's dictation into a |
| | | written report |
| | | Sends/routes report back to |
| | | radiologist for review. |
| | | Makes edits according to |
| | | radiologist's changes. |
| | | Send/routes final report back to |
| | | radiologist for approval. |
| | | Routes final report to referring |
| | | physician and/or appropriate |
| | | parties, if requested. |

An example workflow for a front desk user role or scheduler role, which may correspond in some examples to one of the workflows 108a-n discussed above with regard to FIG. 1, can include the following steps: Schedule Appointment→Verify Insurance Eligibility or Pre-authorization→Send Appointment Reminder→Check in Patient for Exam→Complete Exam. In some simple examples, the steps of the workflow from the Schedule Appointment step through the Check in Patient for Exam step, may be linear, while in other examples there may be non-linear aspects in this portion of the workflow. Between the Check in Patent for Exam and Complete Exam steps, several steps may be performed by other roles, as will be discussed. As discussed above, workflows can be non-linear and can branch depending on any of a variety of factors. The computer system 102 discussed above with regard to FIG. 1 can implement the example workflow for the front desk/scheduler through the use and transmission of appropriate client-side codes to a client computing device for the front desk/scheduler which, when selected, can notify the computer system 102 of the progress of the workflow.

In one example use case of such a workflow, a patient or referring physician can request an appointment. For example, the patient or referring physician may request a diagnostic imaging exam for the patient. In various examples, a referrer or patient can place an order in several ways. For example, the referrer may call an imaging center to schedule an exam or appointment for a patient, or may log in to an online physician portal and request the appointment electronically. As another example, the referrer may refer the patient and the patient may call the center to make an appointment, or log in to a patient portal and request the appointment electronically.

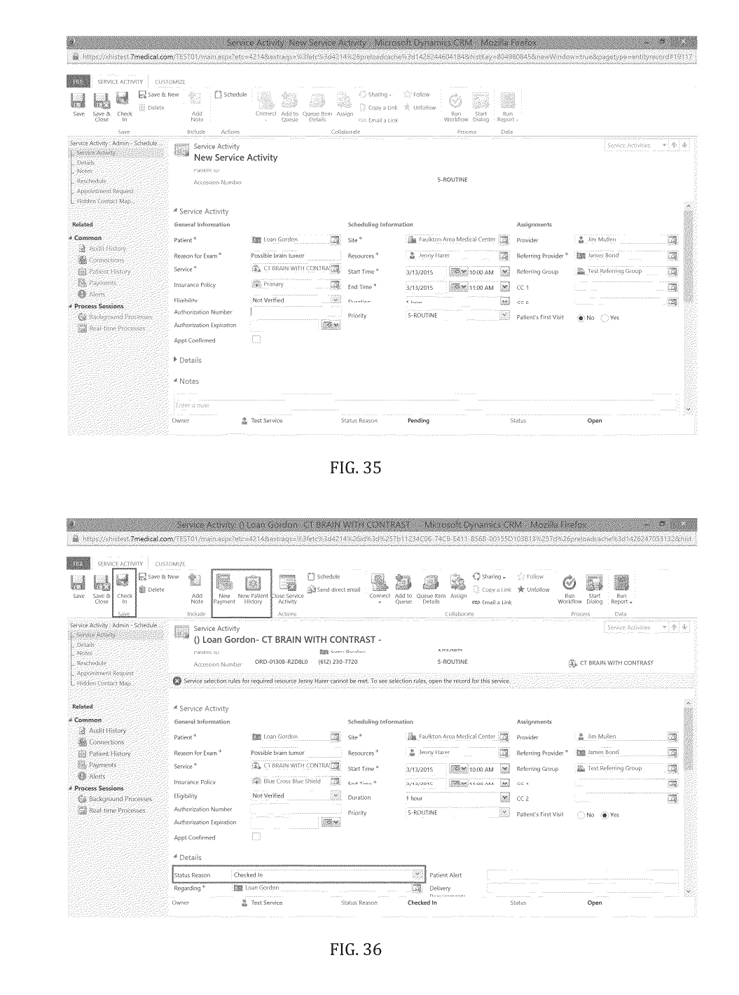

The scheduler can schedule the appointment and, through the use of client-side code features on the scheduler's computing device, can provide information to the computer system 102 indicating that the appointment has been scheduled. FIG. 35 is a screen shot of an example user interface that includes a new service activity, and which a scheduler may use to schedule an appointment, for example. The scheduler can schedule the appointment by creating and filling out the new service activity (appointment) form, which may also create an Order for the exam. The scheduler can select a patient using a lookup tool, which may automatically pull patient insurance information and populate the appropriate fields in the service activity, or can enter new patient information. In some examples, one or more aspects of a patient's insurance policy may influence products and services, or pricing thereof, related to the order, for example.

In general, at each step of the workflow, the client computing device (e.g., the scheduler's client computing device in this example) may provide information to the computer system so that progress of the workflow may be tracked and appropriate next steps of the workflow can be scheduled. In various examples, the computer system may provide appropriate client-side code portions (e.g., JavaScript code) to appropriate client computing devices, and the client computing devices may execute or interpret the client-side code portions and perform actions in response. In some examples, client-side code portions are associated with user interface features provided or presented by the client computing devices, and a selection of the user interface feature may cause the client-side code portion to be executed, which may result in information being provided from the client computing device to the computer system. The computer system can use this information to update a progress of the workflow, for example.

For example, receipt of information can cause the computer system 102 to update the status of the appointment and to track the progress along the workflow (progressing from the Schedule Appointment step to the Verify Insurance Eligibility step). Such progression can cause the computer system 102 to determine, given the status (appointment scheduled, workflow at the insurance verification step), who the appropriate user is to perform the next step and a timeframe within which it is to be completed. Rules associated with the workflow and/or organizational information about user roles can be used to make such a determination. For instance, in some organizations insurance verification can be passed over to a biller. In other examples, the scheduler may verify insurance.

In this example, the scheduler is designated by the computer system 102 for verifying insurance. Accordingly, a task for verifying the insurance of the user who was just scheduled for an appointment can be assigned to the scheduler and an appropriate entry for the task can be added to the scheduler's dashboard. Once the entry is selected, appropriate information for insurance verification can be retrieved from the computer system 102 and appropriate client-side code can be identified and retrieved, and the information and client-side code can be provided to the computing device associated with the scheduler. The client-side code can be executed/interpreted by the scheduler's computer system and a button or other user interface feature can be presented that, when selected by the scheduler, can provide verification to the computer system 102 that insurance eligibility has been verified. In some examples, in addition to the patient's insurance eligibility being checked (which may occur electronically in some examples), a check is made to confirm that the procedure or order has been pre-authorized (and if not, a pre-authorization request can be sent), and this can also occur electronically in some examples. Referring again to FIG. 35, at a “Scheduling Information” section of the user interface, a site can be selected for the exam, as well as a resource, start time, a predicted end time, and a duration based on the start time and the predicted end time. At an “Assignments” section of the user interface, the scheduler can select which users and/or teams have access to view this record (e.g., Provider, Provider Team, Referring Provider, Referring Group).

Similar to the previous step, the computer system 102 can receive the verification information and can progress to the next step in the workflow (Send Appointment Reminder). As with the previous step, the computer system 102 can determine, given the status (insurance verified, workflow at the appointment reminder step), who the appropriate user is to perform the next step and a timeframe within which it is to be completed. Rules associated with the workflow and/or organizational information about user roles can be used to make such a determination.

In this example, the scheduler can be designated as the user responsible for the appointment reminder task and the task can be listed on the scheduler's dashboard. Once the task is selected, appropriate information and client-side code information can be served to the scheduler's computing device by the computer system 102. The scheduler's computing device can execute/interpret the client-side code, which can be different from the other client-side codes that have been provided to the scheduler's client device (e.g., can have different information identifying the step that is being performed/confirmed by the scheduler). Once a client-side code feature is selected by the scheduler, information confirming that the reminder has been sent to the user can be provided to the computer system 102, which can then determine a next step in the workflow and a user to perform it. The scheduler can again be designated to perform for the next step (Check Patient in at Exam) and, when the patient arrives, the scheduler can check the patient in for the exam. When the scheduler selects an entry to check-in the patient, which can be a client-side code feature, information regarding the check-in can be transmitted to the computer system 102, which can then transfer handling of the patient exam to another user (e.g., technologist, nurse, doctor) and which may use one or more other workflows to monitor the patient exam, such as a technologist, nurse and/or doctor workflows for patient exams.

When the patient arrives for the appointment, the scheduler can check the patient in to start the order process, for example. FIG. 36 is a screen shot of an example user interface that a scheduler can use to check in a patient, for example. A service activity—CT Brain with Contrast—is depicted in the GUI of FIG. 36. The user interface of FIG. 36 includes a custom “Check In” button, and is associated with client-side code that may execute when the scheduler's client computing device receives a selection of the Check In button. For example, when the Check In button is selected, the client-side code may execute, and may cause a Status Reason of the service activity to be updated to “Check In” as can be seen in FIG. 36, which depicts the service activity after the user has selected the Check In button. In some examples, the selection of the Check In button can also cause a change in a Status Reason on the Order, and the status reason may change to “Begin Procedure”.

In some examples, the client computing device may transmit to the computer system of an indication that the Check In button has been selected (e.g, based on execution of the client-side code associated with the Check In button), and the computer system may update the Status Reason, for example. For example, the computer system may identify a particular workflow based on, for example, an identity or the user or based on a role of the user. The computer system may determine that a status change is appropriate based on, for example, a current status of the order or of a workflow associated with the order, and may select appropriate client-side code for a next step in the process. The computer system may provide updates to one or more client computing devices, for example. In some examples, the client-side code associated with the button may cause the Status Reason to be updated, and an indication of the update may be provided to the computer system. The computer system may provide updates to one or more client computing devices, for example.

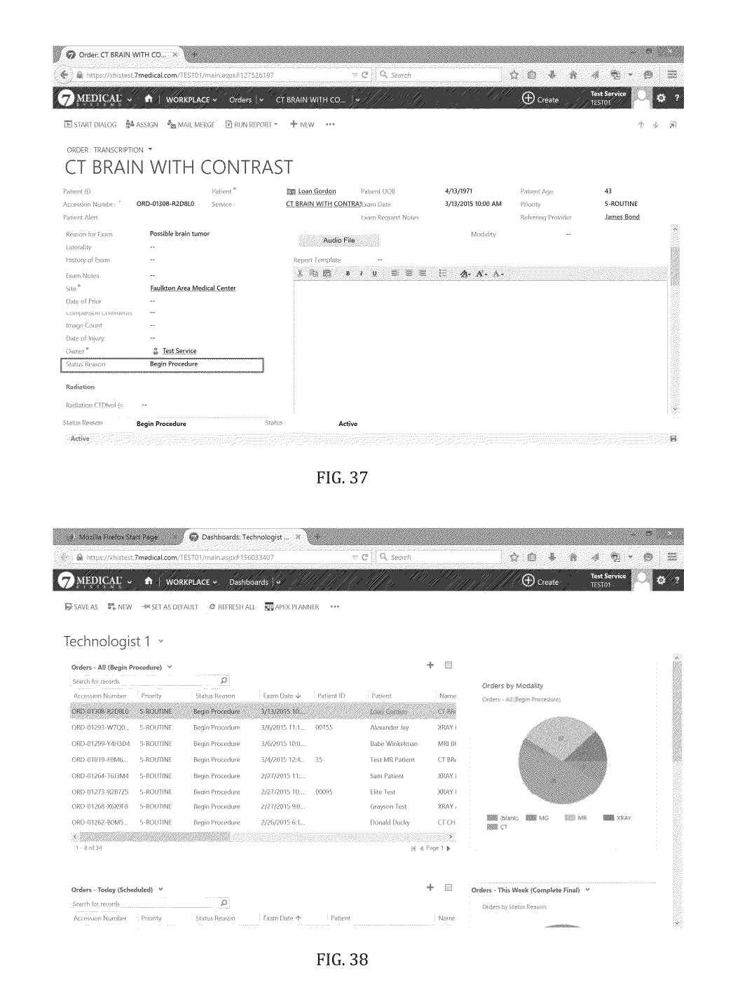

The computer system may use the Status Reason to populate the Order in the worklist of another user in the system, for example. For example, the computer system may populate the Order in the worklist of a Technologist user's dashboard view on the Technologist's client computing device, for example in response to starting an order for the exam. This may alert the Technologist that the patient has been checked in and is waiting or ready for his/her exam, for example. FIG. 38 is a screen shot of an example user interface that includes a dashboard view for a Technologist user role. As can be seen in FIG. 38, orders with a Status reason of “Begin Procedure” are shown. FIG. 37 is a screen shot of an example user interface that includes a dashboard view for a Technologist user role, for example. As can be seen in FIG. 37, the Status Reason is listed as “Begin Procedure.”

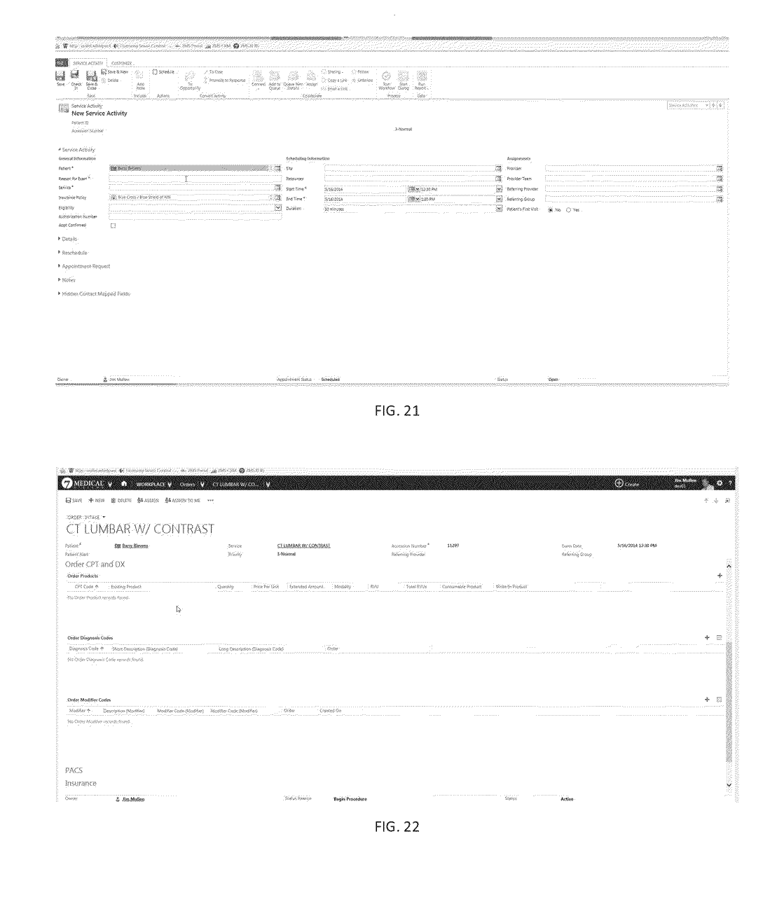

Referring to FIG. 21 (example screenshot), another example user interface through which an appointment (Service Activity) can be scheduled by a scheduler is presented. For example, a scheduler can schedule a new appointment through the following steps: scheduler creates a new Service Activity (appointment), which also creates an Order; select patient or enter new patient info; selecting patient automatically pulls patient's insurance information and populates the insurance fields in the service activity (appointment) and the insurance policy can drive the fee schedule for the products and services related to the order; verify insurance eligibility (electronic verification); if pre-authorization is required, then pre-authorization request is sent/verified; select Site, Resource and times; select which users and teams can view this record; and scheduler performs patient reminder call-downs 24-48 hours prior to scheduled appointment.

Returning to the imaging center example, with the patient checked in and waiting for his or her exam, a first user with whom the patient may meet may be a Technologist, who may be responsible for performing the imaging portion of the patient exam. As described above, FIG. 38 depicts a dashboard for a Technologist, and shows various orders for the Technologist, which may correspond to orders for which the Technologist will conduct an imaging exam for various patients, for example.

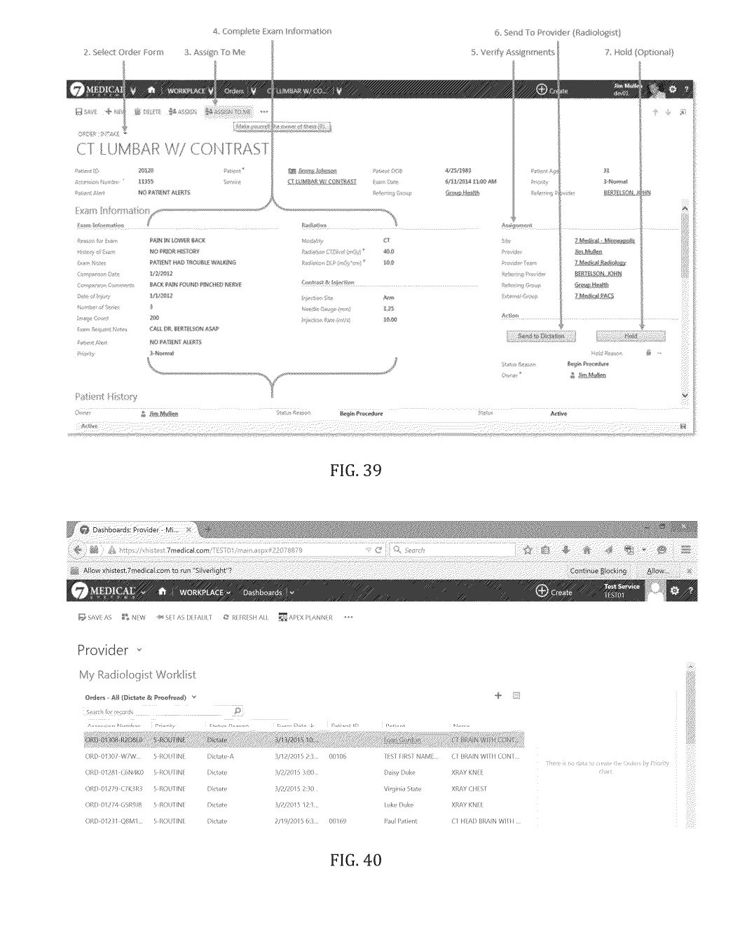

The Technologist may have a separate workflow, or sub-workflow, from the scheduler and other users in the system, for example. An example Technologist workflow (e.g., which may correspond to one of the workflows 108a-n, see FIG. 1) can include the following steps: Open or Create Order→Perform Imaging Exam→Review/Update patient information, order information, and patient clinical information→Assign imaging exam to radiologist or radiology group for interpretation. As an example use case of the workflow, the Technologist may review orders already in the worklist and verify order information, or may create a new order in the system. The Technologist may then perform the imaging exam, and any of a variety of imaging technologies can be used, as appropriate. The Technologist may review and update patient information and history, and may review and update order information and history. The Technologist may review and update patient clinical information. The Technologist may place an order on hold, may request an addendum to an order, or may cancel an order. The Technologist may assign one or more activities regarding the patient or the order (or another order, for example). The Technologist may assign the imaging exam to a radiologist for interpretation, for example, according to business rules. FIG. 39 is a screen shot of an example user interface that a technologist can use to review or enter exam or order information, review or enter patient information, assign the order to another user, or place a hold on the order, for example.

The user interface of FIG. 39 includes a “Send to Dictation” button and a “Hold” button, each of which may have respectively associated therewith client-side code portions that can execute upon receipt of a user selection of the respective button. For example, when the Send to Dictation button is selected, the client-side code may execute, and may cause the Order to be saved, and in some examples may cause a Status Reason of the Order to be updated to “Dictate”. In some examples, the client computing device may transmit to the computer system of an indication that the Send to Dictation button has been selected, and the computer system (rather than the client computing device) may update the Status Reason, for example. The computer system may provide updates to one or more client computing devices, for example. In some examples, the client-side code associated with the button may cause the Status Reason to be updated, and an indication of the update may be provided to the computer system. The computer system may provide updates to one or more client computing devices, for example. For example, the Status Reason of Dictate may cause the computer system to populate the Order in the worklist of a Radiologist Dashboard. This may alert a radiologist that he or she now has an action item, for example. In some examples, all orders having a Status Reason of Dictate will appear in a worklist of a Radiologist Dashboard.

Regarding the Hold button in the user interface of FIG. 39, a Technologist may select the Hold button when there is an issue with an order, and the Technologist wishes to place the order on hold so that it does not advance further in the workflow. When the Hold button is selected, the client-side code may execute, and may cause the Order to be saved, and in some examples may cause a Status Reason of the Order to be updated to “Hold”. In some examples, the client computing device may transmit to the computer system of an indication that the Hold button has been selected, and the computer system (rather than the client computing device) may update the Status Reason, for example. The computer system may provide updates to one or more client computing devices, for example. In some examples, the client-side code associated with the button may cause the Status Reason to be updated, and an indication of the update may be provided to the computer system. The computer system may provide updates to one or more client computing devices, for example. In some examples, the Status Reason of Hold may cause a step-by-step workflow dialog to be run, where the Technologist is permitted to create a follow-up activity (e.g., task or other activity) to address the issue with the Order.

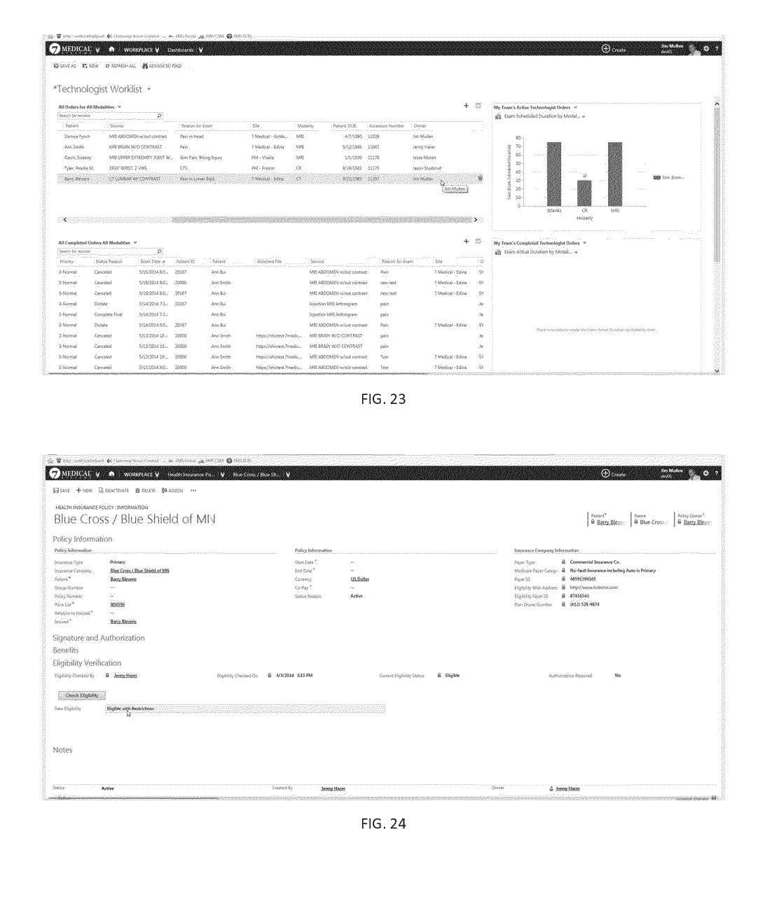

A user interface depicting an example imaging order for a CT Lumbar with Contrast is depicted in FIG. 22 (example screenshot) and an example screenshot of a dashboard for the technologist is depicted in FIG. 23 (example screenshot).

Workflows can be created and used across a variety of other roles as well, such as radiologist roles and transcriptionist roles. For example, when the Technologist selects the Send to Dictation, as described above with reference to FIG. 39, the Order may appear in the worklist of a Radiologist Dashboard view. FIG. 40 is a screen shot of an example user interface that a radiologist can use to view a list of Orders, for example. In some examples, the orders may include a status reason of Dictate, which may indicate that the Radiologist should review one or more images, or Proofread, which may indicate that the Radiologist should proofread a diagnostic report, for example. In some examples, the orders may be sorted in reverse chronological order by exam date, for example.

A workflow for a Radiologist may include: Interpret imaging exam (study)→Dictate diagnostic findings→Proof/approve diagnostic radiology report. An example use case may include the Radiologist viewing imaging exams (studies) in the worklist from the radiology dashboard (e.g., the dashboard shown in FIG. 40). The Radiologist may view an imaging exam and may dictate information regarding the imaging exam using voice recognition software, for example, or may make an audio recording of information regarding the imaging exam to be transcribed by a Transcriptionist, or may type a diagnostic report, for example using preset structured report templates configured in the system. If voice recognition software is used, the Radiologist may proofread the report output of the voice recognition software, and may sign and close the report if it meets expectations. If an audio recording is used, the Radiologist may route the audio recording to a Transcriptionist. If a preset structured report template is used, the Radiologist may proofread the report, and may sign and close the report if it meets expectations. A signed and completed diagnostic radiologist report may be available to a referring physician via a referring physician portal, or may be routed to the referring physician based on rules/preferences (e.g., using online portal, fax, alert).

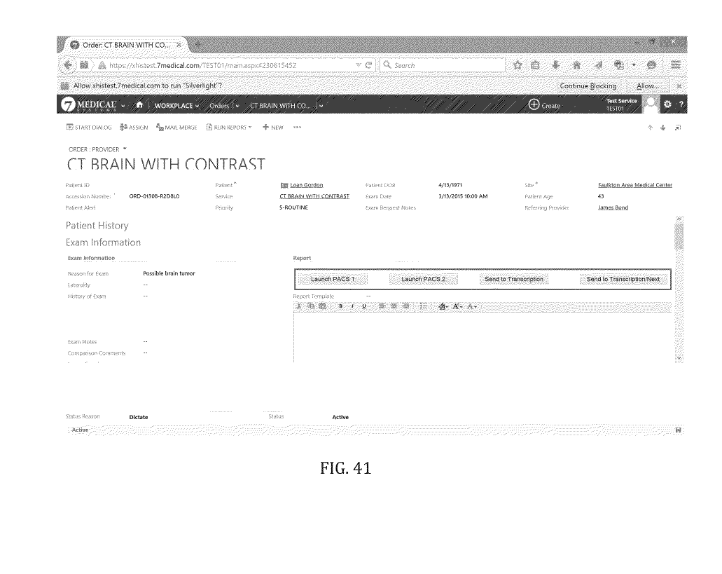

FIG. 41 is a screen shot of an example user interface that a radiologist can use to view details of an Order, for example. For example, the Radiologist may select an Order from the worklist view of FIG. 40, and may be presented with the view of FIG. 41. The Radiologist can interpret a radiologic image and provide findings in a number of ways. The user interface of FIG. 41 includes a “Launch PACS 1” button, a “Launch PACS 2” button, a “Send to Transcription” button, and a “Send to Transcription/Next” button, each of which may have respectively associated therewith client-side code portions that can execute upon receipt of a user selection of the respective button.

For example, when the Launch PACS 1 button is selected, the associated client-side code may execute, and may launch a picture archiving and communications (PACS) system viewer so that the Radiologist can view the image (x-ray, CT or other modality) and dictate. For example, when the Launch PACS 2 button is selected, the associated client-side code may execute, and may launch a secondary PACS viewer. For example, when the Send to Transcription button is selected, the associated client-side code may execute, and may cause the Order to be saved, and in some examples may cause a Status Reason of the Order to be updated to “Transcribe,” and in some examples may populate an Order in the worklist of the Transcriptionist Dashboard. In some examples, the client computing device may transmit to the computer system of an indication that a particular button has been selected, and the computer system (rather than the client computing device) may update the Status Reason or perform the other actions, for example. The computer system may provide updates to one or more client computing devices, for example (e.g., to the client computing device of the Transcriptionist if the Transcriptionist should be alerted to an Order based on a Status Reason change). In some examples, the client-side code associated with the button may cause the Status Reason to be updated or one or more other actions to be performed, and an indication of the update and/or actions may be provided to the computer system. The computer system may provide updates to one or more client computing devices, for example. For example, the Status Reason of Transcribe may cause the computer system to populate the Order in the worklist of a Transcriptionist Dashboard. This may alert a transcriptionist that he or she now has an action item, for example. In some examples, all orders having a Status Reason of Transcribe will appear in a worklist of a Transcriptionist Dashboard. For example, when the Send to Transcription/Next button is selected, the associated client-side code may execute, and may cause the Order to be saved, and in some examples may cause a Status Reason of the Order to be updated to “Transcribe,” and in some examples may populate an Order in the worklist of the Transcriptionist Dashboard, and may open a next Order in the Radiologist worklist.

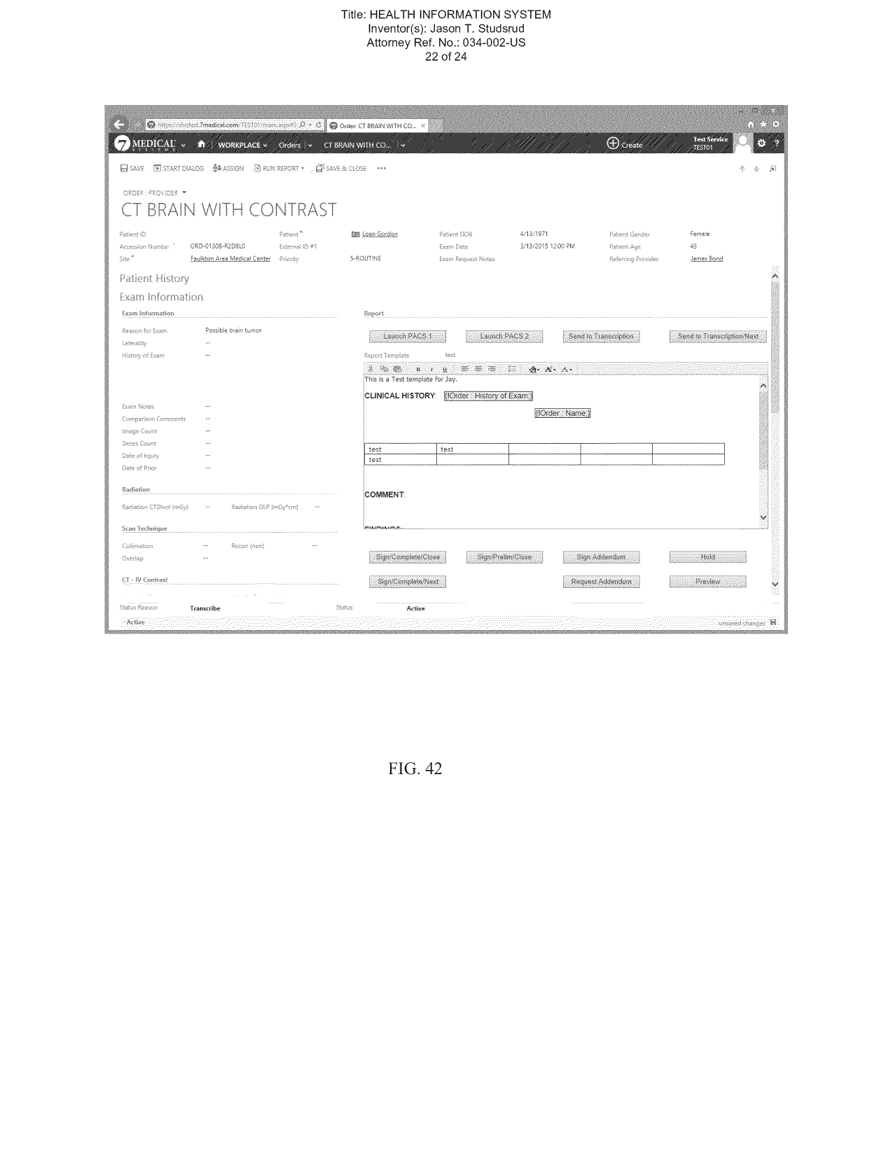

As described above, a Radiologist can use preset structured report templates that are configured in the system. A report template can be selected using a lookup tool, for example, and the text and structured fields for a selected report template can be populated in a Report Editor. FIG. 42 is a screen shot of an example user interface that a radiologist can use to view details of an Order and prepare a report using a structured report template (CT BRAIN in this example), for example. The user interface of FIG. 42 includes a “Sign/Complete/Close” button, a “Sign/Prelim/Close” button, a “Sign/Complete/Next” button, a “Sign Addendum” button, a “Request Addendum” button, a “Hold” button, and a “Preview” button. Each of the buttons may have respectively associated therewith client-side code portions that can execute upon receipt of a user selection of the respective button.

For example, when the Sign/Complete/Close button is selected, the associated client-side code may execute, and the radiologist's digital signature may be added to the report, and the report may be saved and closed. When the Sign/Prelim/Close button is selected, the associated client-side code may execute, and the radiologist's digital signature may be added to the report, and a status of the report may be updated to “preliminary.” When the Sign/Complete/Next button is selected, the associated client-side code may execute, and the radiologist's digital signature may be added to the report, the report may be saved and closed, and a next Order in the radiologist's worklist may be opened. When the Sign Addendum button is selected, the associated client-side code may execute, and the radiologist's digital signature may be added to the addendum, and the Order may be closed. When the Request Addendum button is selected, the associated client-side code may execute, and a workflow dialog may be provided to request an addendum to the existing report. A Status Reason for the Order may be changed to Dictate-A (for dictate addendum), and an order may be populated in the worklist on the Radiologist's dashboard, to alert the Radiologist to the task. When the Hold button is selected, such as because there may be an issue with the order, the associated client-side code may execute, and a workflow dialog to create an activity (task) to address the issue may be presented. When the Preview button is selected, the associated client-side code may execute, and a preview of the report may be presented. In some examples, the client computing device may transmit to the computer system of an indication that a particular button has been selected, and the computer system (rather than the client computing device) may update the Status Reason or perform the other actions, for example. The computer system may provide updates to one or more client computing devices, for example. In some examples, the client-side code associated with the button may cause the Status Reason to be updated or one or more other actions to be performed, and an indication of the update and/or actions may be provided to the computer system. The computer system may provide updates to one or more client computing devices, for example.

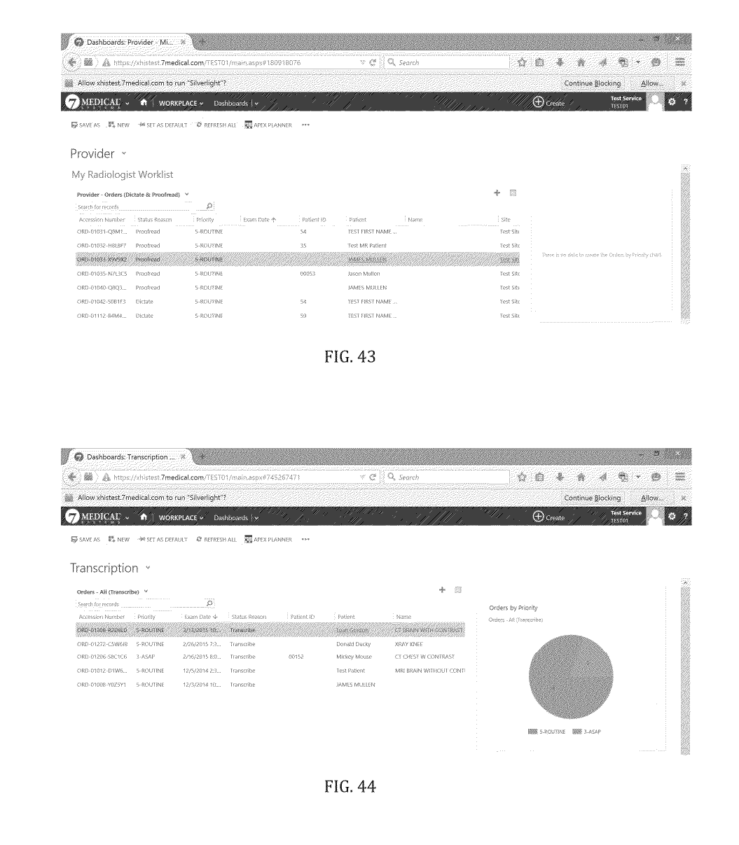

In some examples, the Radiologist may also proofread a report from a transcriptionist. For example, the radiologist may proofread a report prepared by a transcriptionist, who may prepare the report based on an audio recording prepared by the radiologist. FIG. 43 is a screen shot of an example user interface that presents a dashboard view that a radiologist can use to view orders and select an order (e.g., the highlighted order) to proofread, for example. When the radiologist selects the order, a view similar to the view of FIG. 42 may be presented, except that a transcriptionist-prepared report may replace the structured template report. Also included in the view may be the buttons, with the associated functionality, described above with reference to FIG. 42.

Workflows can be created and used across a variety of other roles as well, such as a transcriptionist role. For example, when the Radiologist selects the Send to Transcription button, as described above with reference to FIG. 41, the Order may appear in the worklist of a Transcriptionist Dashboard view, for example based on a change in Status Reason to Transcribe. FIG. 44 is a screen shot of an example user interface (e.g., a transcriptionist dashboard) that a transcriptionist can use to view a list of Orders, for example. A workflow for a Transcriptionist may include: View orders via Transcriptionist worklist→Transcribe radiology report→Route transcribed report to Radiologist for proofreading/sign-off→Route finalized report to referring physician based on rules. An example use case may include the Transcriptionist viewing orders in the queue that are ready to be transcribed (e.g., by viewing the dashboard of FIG. 44), typing or transcribing the radiology report, routing the transcribed report to the corresponding radiologist for review/approval, and routing the finalized report to a referring physician.

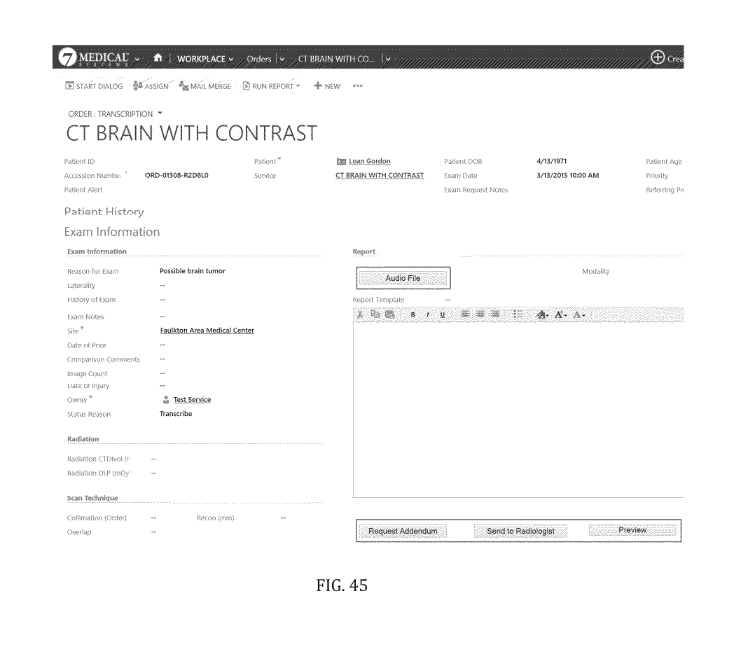

In some examples, the transcriptionist may select an order from the dashboard view, and may be presented with a view as depicted in FIG. 45. FIG. 45 is a screen shot of an example user interface that a transcriptionist can use to transcribe a report, for example. The transcriptionist can listen to an audio recording of the report, and can transcribe the audio report by typing a draft of the report into the report editor.

The user interface of FIG. 45 includes an “Audio File” button, a “Request Addendum” button, a “Send to Radiologist” button, and a “Preview” button, each of which may have respectively associated therewith client-side code portions that can execute upon receipt of a user selection of the respective button.

For example, when the Audio File button is selected, the associated client-side code may execute, and may open an audio file, which may correspond to a dictated radiology report, for example. For example, when the Request Addendum button is selected, the associated client-side code may execute, and a workflow dialog for requesting an addendum may be presented (e.g., a dialog that includes a step-by-step wizard). For example, when the Send to Radiologist button is selected, the associated client-side code may execute, and may cause the Order to be saved, and in some examples may cause a Status Reason of the Order to be updated to “Proofread,” and in some examples may populate an Order in the worklist of the Radiologist Dashboard. In some examples, the client computing device may transmit to the computer system of an indication that a particular button has been selected, and the computer system (rather than the client computing device) may update the Status Reason or perform the other actions, for example. The computer system may provide updates to one or more client computing devices, for example (e.g., to the client computing device of the Radiologist if the Radiologist should be alerted to an Order based on a Status Reason change (e.g., the change to Proofread)). In some examples, the client-side code associated with the button may cause the Status Reason to be updated or one or more other actions to be performed, and an indication of the update and/or actions may be provided to the computer system. The computer system may provide updates to one or more client computing devices, for example. When the Preview button is selected, the associated client-side code may execute, and a preview of the report may be presented.

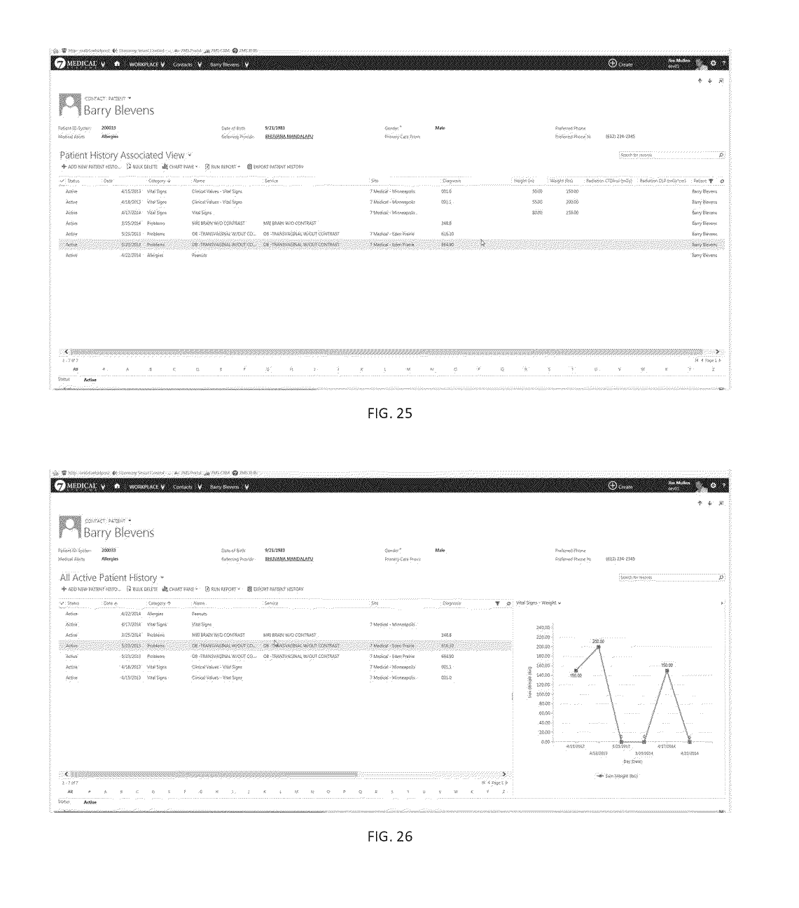

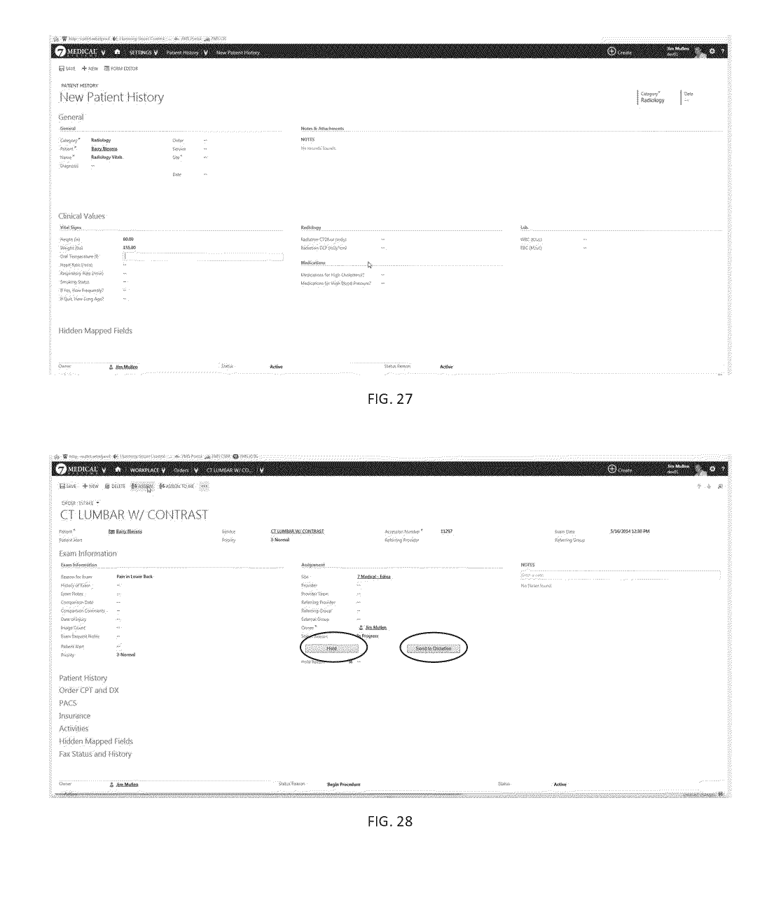

The disclosed computer systems can use a variety of custom entities and scripts to provide the discussed health information systems. For example, the following custom entities can be used: Contact Type (e.g., Patient, Provider, Referring Physician), Insurance Policy (e.g., fields to capture all relevant primary, secondary and tertiary insurance information, FIG. 24 depicts an example Insurance Entity and associated information), Service (e.g., CPT Code, Diagnosis Code, Modifiers), Secure Messaging (e.g., sends secure message to any user in the system, sends SMS text messages to users' mobile phones), Patient History (e.g., used to chart patient notes, used to meet HITECH Act meaningful use (e.g., problem list), forms used to track clinical, radiology and other vital signs, documents can be scanned in and attached to the history record (link to open scanned document is linked via JavaScript)). Example patient history views are presented in FIG. 25 (example problem list) and FIG. 26 (example chart of vital signs), and an example of a new patient history form is presented in FIG. 27.

Example user interface features/controls that can be presented in user interfaces on client computing devices can include, for example, a “Check In” button that is presented as part of a front desk/scheduler workflow, and “Hold” and “Send to Dictation” buttons that are presented as part of a technologist workflow. For example, FIG. 28 depicts these example “Hold” and “Send to Dictation” buttons, which may be presented in the user interface by execution/interpretation of appropriate client-side code from a computer system managing the workflow, such as the computer system 102.

The computer systems and techniques disclosed in this document can additionally and/or alternatively use ICD-9 codes, ICD-10 codes, and/or SNOWMED terms/codes, and can convert between them. For example, the computer systems 102, 202, and/or 302 can convert from ICD-9 to ICD-10 codes, and may use modifier fields associated with such ICD-9 codes and/or ICD-10 codes to resolve any ambiguities that may arise from first code (e.g., single ICD-9 code) mapping to multiple converted codes (e.g., multiple ICD-10 codes). In another example, narratives can be converted into SNOMED, which can then be converted to ICD-10 codes. For instance, SNOMED terms can provide standard concepts and terms that can be mapped to ICD-10 codes.

The computer systems and techniques disclosed in this document can also be used for data analysis to provide business intelligence information, charts and reports.

The computer systems and techniques disclosed in this document can also be used to provide cross-platform compatibility between different data systems. As previously described, for example, the system can enable data exchange between systems by serving as an integration component to provide one-to-one integration, one-to-many integration (or vice versa), or many-to-many integration. For example, the disclosed master databases can include a repository of data mappings that map fields from the master database to one or more other systems, such as EMR and EHR systems. Such mappings can allow for data to more readily be obtained, used, and shared by the disclosed computer systems and techniques, and can help healthcare providers more readily modernize from their older legacy systems and data structures. Additionally, language translations can also be used to translate data from one language (e.g., DICOM, or Digital Imaging and Communications in Medicine) to another language (e.g., HL7, or Health Level-7).

The described systems and methods can be used to automate workflows, and can make a transition of a patient order among actors in a healthcare organization more efficient and seamless, for example. For example, a given order may be worked on by numerous personnel (e.g, users) who may have different roles within the organization as a patient goes through the various steps of requesting an exam and pre-exam processing, partaking in an exam, and the aftermath of the exam. In particular, at various stages of the workflow, different users may be responsible for different tasks or activities associated with the order. In some examples, each of the users may be associated with a client computing device, upon which user interfaces that include features (e.g., selectable buttons) associated with client-side code portions (e.g., JavaScript code scripts) provided by a central computer system. In some examples, a status or status reason of the order may be changed as the order moves through the workflow, and the change may be as a result of execution of the client-side code portions following, for example, a selection of a client side code feature (e.g., a button associated with the client-side code). In this manner, the order may be moved through a process. At a given stage in a workflow, a status or status reason may be common for a given order across the views of the order provided to all of the users, including users having different roles, in some examples. Different information may be presented to users having different roles, in some examples.

Computing devices and computer systems described in this document that may be used to implement the systems, techniques, machines, and/or apparatuses can operate as clients and/or servers, and can include one or more of a variety of appropriate computing devices, such as laptops, desktops, workstations, servers, blade servers, mainframes, mobile computing devices (e.g., PDAs, cellular telephones, smartphones, and/or other similar computing devices), computer storage devices (e.g., Universal Serial Bus (USB) flash drives, RFID storage devices, solid state hard drives, hard-disc storage devices), and/or other similar computing devices. For example, USB flash drives may store operating systems and other applications, and can include input/output components, such as wireless transmitters and/or USB connector that may be inserted into a USB port of another computing device.

Such computing devices may include one or more of the following components: processors, memory (e.g., random access memory (RAM) and/or other forms of volatile memory), storage devices (e.g., solid-state hard drive, hard disc drive, and/or other forms of non-volatile memory), high-speed interfaces connecting various components to each other (e.g., connecting one or more processors to memory and/or to high-speed expansion ports), and/or low speed interfaces connecting various components to each other (e.g., connecting one or more processors to a low speed bus and/or storage devices). Such components can be interconnected using various busses, and may be mounted across one or more motherboards that are communicatively connected to each other, or in other appropriate manners. In some implementations, computing devices can include pluralities of the components listed above, including a plurality of processors, a plurality of memories, a plurality of types of memories, a plurality of storage devices, and/or a plurality of buses. A plurality of computing devices can be connected to each other and can coordinate at least a portion of their computing resources to perform one or more operations, such as providing a multi-processor computer system, a computer server system, and/or a cloud-based computer system.

Processors can process instructions for execution within computing devices, including instructions stored in memory and/or on storage devices. Such processing of instructions can cause various operations to be performed, including causing visual, audible, and/or haptic information to be output by one or more input/output devices, such as a display that is configured to output graphical information, such as a graphical user interface (GUI). Processors can be implemented as a chipset of chips that include separate and/or multiple analog and digital processors. Processors may be implemented using any of a number of architectures, such as a CISC (Complex Instruction Set Computers) processor architecture, a RISC (Reduced Instruction Set Computer) processor architecture, and/or a MISC (Minimal Instruction Set Computer) processor architecture. Processors may provide, for example, coordination of other components computing devices, such as control of user interfaces, applications that are run by the devices, and wireless communication by the devices.

Memory can store information within computing devices, including instructions to be executed by one or more processors. Memory can include a volatile memory unit or units, such as synchronous RAM (e.g., double data rate synchronous dynamic random access memory (DDR SDRAM), DDR2 SDRAM, DDR3 SDRAM, DDR4 SDRAM), asynchronous RAM (e.g., fast page mode dynamic RAM (FPM DRAM), extended data out DRAM (EDO DRAM)), graphics RAM (e.g., graphics DDR4 (GDDR4), GDDR5). In some implementations, memory can include a non-volatile memory unit or units (e.g., flash memory). Memory can also be another form of computer-readable medium, such as magnetic and/or optical disks.

Storage devices can be capable of providing mass storage for computing devices and can include a computer-readable medium, such as a floppy disk device, a hard disk device, an optical disk device, a Microdrive, or a tape device, a flash memory or other similar solid state memory device, or an array of devices, including devices in a storage area network or other configurations. Computer program products can be tangibly embodied in an information carrier, such as memory, storage devices, cache memory within a processor, and/or other appropriate computer-readable medium. Computer program products may also contain instructions that, when executed by one or more computing devices, perform one or more methods or techniques, such as those described above.

High speed controllers can manage bandwidth-intensive operations for computing devices, while the low speed controllers can manage lower bandwidth-intensive operations. Such allocation of functions is exemplary only. In some implementations, a high-speed controller is coupled to memory, display 616 (e.g., through a graphics processor or accelerator), and to high-speed expansion ports, which may accept various expansion cards; and a low-speed controller is coupled to one or more storage devices and low-speed expansion ports, which may include various communication ports (e.g., USB, Bluetooth, Ethernet, wireless Ethernet) that may be coupled to one or more input/output devices, such as keyboards, pointing devices (e.g., mouse, touchpad, track ball), printers, scanners, copiers, digital cameras, microphones, displays, haptic devices, and/or networking devices such as switches and/or routers (e.g., through a network adapter).

Displays may include any of a variety of appropriate display devices, such as TFT (Thin-Film-Transistor Liquid Crystal Display) displays, OLED (Organic Light Emitting Diode) displays, touchscreen devices, presence sensing display devices, and/or other appropriate display technology. Displays can be coupled to appropriate circuitry for driving the displays to output graphical and other information to a user.

Expansion memory may also be provided and connected to computing devices through one or more expansion interfaces, which may include, for example, a SIMM (Single In Line Memory Module) card interfaces. Such expansion memory may provide extra storage space for computing devices and/or may store applications or other information that is accessible by computing devices. For example, expansion memory may include instructions to carry out and/or supplement the techniques described above, and/or may include secure information (e.g., expansion memory may include a security module and may be programmed with instructions that permit secure use on a computing device).

Computing devices may communicate wirelessly through one or more communication interfaces, which may include digital signal processing circuitry when appropriate. Communication interfaces may provide for communications under various modes or protocols, such as GSM voice calls, messaging protocols (e.g., SMS, EMS, or MMS messaging), CDMA, TDMA, PDC, WCDMA, CDMA2000, GPRS, 4G protocols (e.g., 4G LTE), and/or other appropriate protocols. Such communication may occur, for example, through one or more radio-frequency transceivers. In addition, short-range communication may occur, such as using a Bluetooth, Wi-Fi, or other such transceivers. In addition, a GPS (Global Positioning System) receiver module may provide additional navigation- and location-related wireless data to computing devices, which may be used as appropriate by applications running on computing devices.

Computing devices may also communicate audibly using one or more audio codecs, which may receive spoken information from a user and convert it to usable digital information. Such audio codecs may additionally generate audible sound for a user, such as through one or more speakers that are part of or connected to a computing device. Such sound may include sound from voice telephone calls, may include recorded sound (e.g., voice messages, music files, etc.), and may also include sound generated by applications operating on computing devices.

Computing devices can also include one or more sensors through which various states of and around the computing devices can be detected. For example, computing devices can include one or more accelerometers that can be used to detect motion of the computing devices and details regarding the detected motion (e.g., speed, direction, rotation); one or more gyroscopes that can be used to detect orientation of the computing devices in 3D space; light sensors that can be used to detect levels of ambient light at or around the computing devices; touch and presence sensors that can be used to detect contact and/or near-contact with one or more portions of the computing devices; environmental sensors (e.g., barometers, photometers, thermometers) that can detect information about the surrounding environment (e.g., ambient air temperature, air pressure, humidity); other motion sensors that can be used to measure acceleration and rotational forces (e.g., gravity sensors, rotational vector sensors); position sensors that can be used to detect the physical position of the computing devices (e.g., orientation sensors, magnetometers), and/or other appropriate sensors.

Various implementations of the systems, devices, and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications, or code) can include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., LCD display screen, LED display screen) for displaying information to users, a keyboard, and a pointing device (e.g., a mouse, a trackball, touchscreen) by which the user can provide input to the computer. Other kinds of devices can be used to provide for interaction with a user as well; for example, feedback provided to the user can be any form of sensory feedback (e.g., visual feedback, auditory feedback, and/or tactile feedback); and input from the user can be received in any form, including acoustic, speech, and/or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

The above description provides examples of some implementations. Other implementations that are not explicitly described above are also possible, such as implementations based on modifications and/or variations of the features described above. For example, the techniques described above may be implemented in different orders, with the inclusion of one or more additional steps, and/or with the exclusion of one or more of the identified steps. Additionally, the steps and techniques described above as being performed by some computing devices and/or systems may alternatively, or additionally, be performed by other computing devices and/or systems that are described above or other computing devices and/or systems that are not explicitly described. Similarly, the systems, devices, and apparatuses may include one or more additional features, may exclude one or more of the identified features, and/or include the identified features combined in a different way than presented above. Features that are described as singular may be implemented as a plurality of such features. Likewise, features that are described as a plurality may be implemented as singular instances of such features. The drawings are intended to be illustrative and may not precisely depict some implementations. Variations in sizing, placement, shapes, angles, and/or the positioning of features relative to each other are possible.

What is claimed is:

1. A computer-implemented method comprising:
receiving, at a computer system implementing a health information system using an underlying customer relationship management (CRM) system, a request for a form related to a particular medical order, the request including a user identifier for a first user requesting the form;
identifying, by the computer system, a role of the first user based on the user identifier;
identifying a particular workflow from among a plurality of workflows based on the medical order and the role of the first user;
determining a current status of the medical order along the particular workflow;
selecting, based on, at least, the current status and the particular workflow, a particular portion of client-side code from among a plurality of portions of client-side code, the particular portion of client-side code being configured to be executed or interpreted by a client computing device associated with the first user to implement a portion of the particular workflow on the client computing device; and
transmitting, by the computer system, the particular portion of client-side code and other information related to the medical order to the client computing device.

2. The computer-implemented method of claim 1, further comprising:
receiving, after transmitting the particular portion of client-side code to the client computing device, an indication that the portion of the workflow was performed on the client computing device;
determining, in response to receiving the indication, a new current status of the particular workflow and the medical order based on the indication.

3. The computer-implemented method of claim 2, further comprising causing an updated status to be presented on a display screen of the client computing device based on the new current status.

4. The computer-implemented method of claim 2, further comprising identifying, based on the new current status and the particular workflow, a next step for the medical order and a second user to perform the next step, wherein the second user is different from the first user.

5. The computer-implemented method of claim 4, further comprising:
selecting, based on, at least, the next step and the second user, a second portion of client-side code from among the plurality of portions of client-side code, the second portion of client-side code being configured to be executed or interpreted by a second client computing device associated with the second user to implement a portion of the particular workflow on the second client computing device; and
transmitting, by the computer system, the second portion of client-side code and other information related to the medical order to the second client computing device.

6. The computer-implemented method of claim 2, wherein the particular workflow includes a sequence of steps in a linear order, and wherein the new current status represents a non-linear variation from the sequence of steps in the linear order.

7. The computer-implemented method of claim 2, further comprising causing a second workflow, different from the particular workflow, to be initiated or resumed.

8. The computer-implemented method of claim 1, wherein the role of the first user is identified from among the group of roles consisting of scheduler, receptionist, nurse, technologist, doctor, transcriptionist, and biller.

9. The computer-implemented method of claim 8, wherein the role of doctor is identified from among the group of roles consisting of radiologist, cardiologist, and neurologist.

10. The computer-implemented method of claim 1, wherein the underlying CRM system is a Microsoft CRM system.

11. The computer-implemented method of claim 1, wherein the particular portion of client-side code is a JavaScript portion of client-side code.

12. The computer-implemented method of claim 1, wherein the particular portion of client-side code is configured, when executed or interpreted by the client computing device associated with the first user, to update a dashboard on a display screen of the client computing device.

13. The computer-implemented method of claim 12, wherein the updating the dashboard includes updating a list of one or more tasks assigned to the first user.

14. The computer-implemented method of claim 13, wherein the one or more tasks are grouped by task type.

15. The computer-implemented method of claim 1, wherein the particular portion of client-side code is configured to be associated with a user interface feature that is presented on a display screen of the client computing device, wherein the particular portion of client-side code is executed in response to a selection of the user interface feature that is presented on the display screen of the client computing device, and wherein the execution of the particular portion of client-side code causes one or more actions or operations to occur.

16. The computer-implemented method of claim 15, wherein the user interface feature is a selectable button.

17. The computer-implemented method of claim 1, wherein the particular portion of client-side code is not included in the underlying CRM system.

18. The computer-implemented method of claim 1, further comprising providing integration between the computer system and one or more remote computer systems.

19. A computer-implemented method comprising:
receiving, at a computer system implementing a health information system using an underlying customer relationship management (CRM) system, a request for a form related to a particular medical order, the request including a user identifier for a first user requesting the form;
identifying, by the computer system, a role of the first user based on the user identifier;
identifying a particular workflow from among a plurality of workflows based on the medical order and the role of the first user;
determining a current status of the medical order along the particular workflow;
selecting, based on, at least, the current status and the particular workflow, a particular portion of client-side code from among a plurality of portions of client-side code, the particular portion of client-side code being configured to be executed or interpreted by a client computing device associated with the first user to implement a portion of the particular workflow on the client computing device;
transmitting, by the computer system, the particular portion of client-side code and other information related to the medical order to the client computing device;
receiving, after transmitting the particular portion of client-side code to the client computing device, an indication that the portion of the workflow was performed on the client computing device;
determining, in response to receiving the indication, a new current status of the particular workflow and the medical order based on the indication;
identifying, based on the new current status and the particular workflow, a next step for the medical order and a second user to perform the next step, wherein the second user is different from the first user;
selecting, based on, at least, the next step and the second user, a second portion of client-side code from among the plurality of portions of client-side code, the second portion of client-side code being configured to be executed or interpreted by a second client computing device associated with the second user to implement a portion of the particular workflow on the second client computing device; and
transmitting, by the computer system, the second portion of client-side code and other information related to the medical order to the second client computing device.

20. The computer-implemented method of claim 19, wherein the particular workflow includes a sequence of steps in a linear order, and wherein the new current status represents a non-linear variation from the sequence of steps in the linear order.
